(19) United States
(12) Patent Application Publication
(10) Pub. No.: US 2026/0345015 A1
(43) Pub. Date: Nov. 5, 2026

(54) SYSTEMS, DEVICES, AND METHODS FOR VISUALIZING PATIENT PHYSIOLOGICAL DATA

(71) Applicant: JRAS MEDICAL INC., ST. JOHN'S (CA)

(72) Inventors: ANDREW J. SMITH, VANCOUVER (CA); GERARD NOSEWORTHY, CONCEPTION BAY SOUTH (CA); EBRAHIM KARAMI, ST. JOHN'S (CA); CHRISTIAN ANDERSON TELLEZ CASTRO, ST. JOHN'S (CA); MARCUS FORD, PORTUGAL COVE-ST. PHILIPS (CA)

(21) Appl. No.: 19/124,773

(22) PCT Filed: Oct. 26, 2023
(86) PCT No.: PCT/CA2023/051430

§ 371 Date: Apr. 25, 2025

Related U.S. Application Data
(60) Provisional application No. 63/419,668, filed on Oct. 26, 2022.

(51) Int. Cl. A61B 5/021; A61B 5/00; A61B 5/0205; A61B 5/024; A61B 5/107; A61B 90/00

(52) U.S. Cl. CPC A61B 5/02108; A61B 5/0205; A61B 5/6823; A61B 5/6835; A61B 5/7278; A61B 5/7285; A61B 5/7485; A61B 5/02416; A61B 5/1071; A61B 5/1079; A61B 2090/3908; A61B 2090/3937; A61B 2576/00

(57) ABSTRACT

Systems, devices, and methods described herein enable generation of images and other visual elements for assisting a clinician in evaluating one or more physiological conditions, including, for example, cardio-respiratory conditions of a patient. In some embodiments, a composite image is generated that includes a visual image of a target portion of a user's body, such as the neck, which is overlaid with visual elements indicative of a patient's physiological information. Images of the target portion are obtained using a sensing device positionable on the patient in a pre-determined position and orientation via a positioning element that uniquely fits with a patient anatomical feature. The images are processed to extract spatially resolved signals within multiple subregions. The signals are processed using synchronizing data spanning multiple physiological cycles to identify waveform segments, which are processed to generate a visual indication representative of the waveform segments, which is rendered in the composite image.

### CROSS-REFERENCE TO RELATED APPLICATION

This application claims priority to U.S. Provisional Patent Application No. 63/419,668, titled “SYSTEMS, DEVICES, AND METHODS FOR VISUALIZING PATIENT PHYSIOLOGICAL DATA” and filed on Oct. 26, 2022, the entire contents of which is incorporated herein by reference.

### TECHNICAL FIELD

Embodiments describe herein relate to systems and methods for generating visual data indicative of a patient's physiological condition and particularly, the patient's cardiac, respiratory, and/or cardiorespiratory condition of a patient to facilitate a medical professional in visually evaluating a patient's physiological condition.

### BACKGROUND

Jugular venous pressure (JVP) or JVP height is a clinical sign evaluated for various applications, including assessment of heart failure (HF) and assessment of volumetric load on the heart.

The internal jugular vein (IJV) descends from the angle of the mandible to the middle of the clavicle at the posterior border of the sternocleidomastoideole. It transmits blood from the brain, skull, face, and neck to the superior vena-cava and ultimately to the right atrium of the heart. The external jugular vein (EJV), located in the anterior and lateral neck, receives blood from the deeper parts of the face as well as the scalp. The external jugular vein starts in the parotid at the level of the angle of the mandible and runs vertically down the neck along the posterior border of the sternocleidomastoideole. At its distal end, the external jugular vein perforates the deep neck fascia and terminates in the subclavian vein. By observing the blood column engorging the IJV and/or the EJV when the body, head and neck are at a specific angle to the horizontal, an assessment of the JVP and the right atrial pressure can be made. Evaluation of the JVP is a standard procedure in a clinical setting carried out by a physician as part of the physical examination of a patient. During examination, the vertical distance between the sternal angle and a top of the pulsation point of the IJV (or EJV) is measured. In some instances, a hepatojugular reflux test may be performed, e.g., by pressing the right upper quadrant of the abdomen, atop the liver, to cause more blood to rush into the right atrium and the JVP to increase. The hepatojugular reflux can be used to validate the point of jugular distension (e.g., by noting the upward movement of the IJV or EJV in response).

To measure the distance between the sternal angle and the top of the pulsation point of the IJV, a horizontal line is made from the highest IJV pulsation point to intersect a vertical line, which is erected perpendicular to the ground through the sternal angle of Louis. Typically, these “lines” are made usually of readily available straight objects, such as rulers. The distance between the sternal angle and the intersection of the two lines is measured along the vertical line. The sum of this measured distance plus 5 cm, which is obligatorily added owing to the fixed relationship between the outer surface of the patient's body and the midpoint of the right atrium (if measured when the patient is at a 30-degree angle), represents the patient's mean JVP. For example, FIG. 1A shows a diagram 10 of a patient lying on a bed at a reclination angle A (e.g., in a range of about 30 degrees to about 60 degrees). The vertical distance between the sternal angle (H_{SA}) and the height of the blood column in the IJV (H_{IJV})+5 cm is the JVP (H_{JVP} in FIG. 1A). It is noted that some clinicians may report the JVP as the height above the right atrium, adding a 5 cm that represents the average height offset between the sternal angle and the right atrium), while other clinicians may report the JVP height as the height above the sternal angle.

The normal mean jugular venous pressure, determined as described above (i.e., 5 cm+the vertical height in cm above the sternal angle), is 6 to 8 cm H₂O. Deviations from this normal range can reflect, for example, hypovolemia (i.e., where the mean venous pressure is less than, for example, about 5 cm H₂O) or hypervolemia (i.e., where the mean venous pressure is greater than, for example, about 8 cm H₂O). Accordingly, a JVP height of less than 7 cm (2 cm above sternal angle) can suggest hypovolemia and greater than 10 cm (5 cm above sternal angle) can suggest hypervolemia. An elevated JVP assessed by a trained physician can suggest early volume overload, predict clinical deterioration and assist with therapeutic decisions in HF and other medical conditions such as renal failure.

Thus, it is desirable to have systems and methods that can generate visual data indicative of a patient's vascular pulsatile motion or other physiological data (e.g., cardiac, respiratory, or cardiorespiratory data) that can be used by a medical professional or caregiver in assessing a patient, e.g., to estimate the patient's JVP or other physiological data therefrom.

### SUMMARY

Systems, devices, and methods described herein enable generation of images and other visual elements for assisting a clinician in evaluating one or more physiological parameters associated with the cardio-respiratory status of the patient, including, for example, the JVP height. In some embodiments, a composite image is generated that includes a visual image of a target portion of a user's body, such as the neck, which is overlaid with visual elements indicative of a patient's physiological information. Images of the target portion are obtained using a sensing device positionable on the patient in a pre-determined position and orientation via a positioning element uniquely fits with a patient anatomical feature. The images are processed to extract spatially resolved signals within multiple subregions. The signals are processed using synchronizing data spanning multiple physiological cycles to identify waveform segments, which are processed to generate a visual indication representative of the waveform segments, which is rendered in the composite image.

Accordingly, in some embodiments, systems, devices, and methods described herein enable generation of visual information including an image of a target portion of a user's body, such as a neck, which is overlaid with spatially segmented and temporally representative (e.g., averaged) visual indications of a patient's physiological data obtained using electromagnetic signals measured by a sensing system. In particular, the target portion of the user's body can be spatially segmented or broken into multiple areas or regions, and representative waveforms over at least a portion of a temporal cycle of the physiological data associated with each underlying area or region (e.g., pulsatile motion or other repetitive motion of the underlying tissue) can be provided over each area or region. In other words, the visual indications provided in each spatial segment of the physiological parameter display corresponds to the physiological data measured in a corresponding spatial area or region of the target portion of the user's body. In some embodiments, this information can enable a medical professional or caregiver to estimate or determine from the visual information, via visual observation, one or more physiological conditions of the patient, including, for example, the JVP or any other cardiorespiratory parameter.

Many physiologic signals (e.g. vascular pulsation, respiration; irrespective of how they are measured) generate tissue motion and are pseudo-periodic in nature. Being able to extract, represent and display a representative waveform (or portions thereof) of the recurring signals in a concise format (e.g. image) is of considerable value. Tissue motion associated with general movement (head turning, speaking) is considered noise. It is generally non-periodic and impedes measurement and display of the underlying signal of interest. Various example embodiments of the present disclosure facilitate the removal of such extraneous tissue motion to facilitate an improvement in signal-to-noise ratio when detecting a pseudo-periodic physiological signal via imaging.

In some embodiments, a control unit for displaying a physiological parameter of a patient is configured to receive one or more images of neck of the patient obtained over a period of time, and receive a synchronizing data (e.g., electrocardiogram (ECG) data or photoplethysmography (PPG) data) of the patient over the period of time, the synchronizing data including or associated with a plurality of cardiac cycles. The control unit is configured to overlay physiological data on an image of the neck to form a composite image, the data being divided into a plurality of spatial nodes or regions (e.g. subregions). The control unit is configured to determine an optical signal of each node (subregion) of the plurality of nodes (subregions) over the period of time, synchronize the optical signal associated with each node (subregion) with the synchronizing data over the period of time, and analyze and/or process the determined optical signal in each node (subregion) during at least a portion of each cardiac cycle in the period of time over the number of cardiac cycles to obtain a representative waveform. The control unit is configured to generate a signal configured to display the composite image such that each node (subregion) of the region of interest in the composite image includes an indication of the representative waveform for that node (subregion) over a least the portion of the cardiac cycle, the indication in each node (subregion) indicative of an average physiological parameter of the patient in a portion of the neck of the patient underlying a respective node (subregion) of the region of interest.

In some embodiments, a method includes: receiving a series of images of a neck of the patient obtained over a period of time, receiving synchronizing data of the patient over the period of time, the synchronizing data including a plurality of cycles, determining, for each node of a plurality of nodes in a region of interest of the neck of the patient, a signal associated with pixel intensities in the series of images, synchronizing the signal for each node of the plurality of nodes with the synchronizing data, segmenting the signal for each node of the plurality of nodes into individual waveforms each corresponding to a different cycle of the plurality of cycles, analyze the individual waveforms for each node of the plurality of nodes to obtain a representative waveform, and generating a composite image including an image of the region of interest and an indication of the representative waveform for each node of the plurality of nodes for at least a portion of a time window associated with the cycle, the indication of the representative waveform for each node of the plurality of nodes being overlaid in an area of the image corresponding to that node.

While the systems, devices, and methods described herein are largely described with reference to determining and generating visual images indicative of the JVP of a patient, it should be understood that such systems, devices and methods are not limited to determining and displaying JVP but can also be used for visualizing other physiological conditions of a patient, including, for example, heart rate, respiratory rate, blood pressure trends, oxygen saturation or carotid (arterial) pulsations. Other embodiments of the present technology are suitable for displaying respiratory effort (e.g., normal and/or distressed). In some embodiments, systems, devices, and methods described herein may be used for monitoring and visualizing physiological data of patients associated with the following conditions: congestive heart failure (CHF), chronic obstructive pulmonary disease (COPD), combinations of both CHF and COPD, asthma, dialysis (e.g., peritoneal and hemodialysis). Further, some embodiments of the present technology may be used in monitoring and visualizing physiological data patients with the following conditions: pericardial tamponade, conditions resulting in elevated intracardiac pressures, scenarios in which excess circulating blood volume is an issue (e.g., sceptic shock after volume resuscitation with IV fluid.), etc.

Accordingly, in a first aspect, there is provided an apparatus, comprising:
a sensing device comprising:
   a positioning element configured to fit within a suprasternal notch of a patient and facilitate alignment of the sensing device with the patient; and
   an imaging assembly rigidly coupled to and spaced from the positioning element, the imaging assembly configured to capture a series of images of at least a portion of a neck of the patient when the positioning element is contacted with the suprasternal notch and aligned with the patient;
processing hardware operatively coupled to the sensing device, the processing hardware comprising a memory and a processor, the memory comprising instructions executable by the processor for performing operations comprising:
   receive the series of images of the neck of the patient obtained over a period of time;
   receive synchronizing data of the patient over the period of time, the synchronizing data spanning a plurality of cardiac cycles;
   define, within the series of images, a region of interest comprising at least a portion of the neck of the patient;
   for each subregion of a plurality of subregions defined within the region of interest:
      generate a time-dependent signal associated with pixel intensities within the subregion in the series of images;
      employ features within the synchronizing data to identify, within the time-dependent signal, a plurality of waveform segments, each waveform segment corresponding to a different cycle; and
   determine, based on user input or sensor data, an inclination angle of the patient;
   process at least one of the series of images and the inclination angle to determine vertical height values corresponding to a plurality of locations within the region of interest;
generate a composite image comprising:
   an image comprising the region of interest;
   for at least one subregion:
      an indication characterizing at least one attribute of the waveform segments associated with the subregion;
      the indication being rendered in spatial association with the subregion; and
   the vertical height values, displayed within the region of interest, thereby facilitating a determination of the vertical height corresponding to each indication.

In some example implementations of the apparatus, the processing hardware is configured such that for the at least one subregion, the indication is generated by processing the waveform segments corresponding to the subregion to obtain a representative waveform segment for the subregion; and employing the representative waveform segment to generate the indication.

In some example implementations of the apparatus, the processing hardware is configured such that the representative waveform segment for each subregion is indicative of a physiological parameter of the patient in a portion of the neck of the patient underlying the subregion.

In some example implementations of the apparatus, the processing hardware is configured such the physiological parameter is associated with an average vascular pulsatile motion of the patient in the portion of the neck of the patient underlying the respective subregion.

In some example implementations of the apparatus, the processing hardware is configured such the indication for each subregion is visually rendered to indicate a degree that the representative waveform segment represents the waveform segments for each subregion.

In some example implementations of the apparatus, the processing hardware is configured such that for at least one subregion, the indication comprises a visual representation of a phase characterizing the waveform segments associated with the subregion.

In some example implementations of the apparatus, the processing hardware is configured such that for at least one subregion, the indication comprises visual representation of an amplitude characterizing the waveform segments associated with the subregion.

In some example implementations of the apparatus, the processing hardware is configured such that for at least one subregion, the indication corresponds to a timing of ventricular contraction relative to the waveform segments associated with the subregion.

In some example implementations of the apparatus, the processing hardware is configured such that, for at least one subregion, the indication is generated by processing the waveform segments associated with the subregion to determine a respective entropy measure associated with each waveform segment; and excluding a subset of the waveform segments that fail to satisfy entropy criteria when generating the indication corresponding to the subregion.

In some example implementations of the apparatus, the processing hardware is configured such that, for at least one subregion, the indication is generated by processing the waveform segments associated with the subregion to determine a subset of waveform segments satisfying similarity criteria; and excluding waveform segments that are not members of the subset when generating the indication corresponding to the subregion.

In some example implementations of the apparatus, the processing hardware is further configured to:
process at least one of the series of images to identify a fiducial marker residing proximal to an ear lobe of the patient;
employ the location of the fiducial marker, and a pre-determined reference location, to construct a reference line associated with a location of an internal jugular vein; and
employ the reference line when generating the region of interest.

In some example implementations of the apparatus, the processing hardware is further configured to:
process at least one of the series of images to identify a fiducial marker residing proximal to an ear lobe of the patient;
employ the location of the fiducial marker, and a pre-determined reference location, to construct a reference line associated with a location of an internal jugular vein; and
employ the reference line when displaying the vertical height values.

In some example implementations of the apparatus, the processing hardware is further configured to:
process the waveform segments associated with each subregion to identify one or subregions associated with pulsations of an internal jugular vein;
identify a highest subregion on the neck that is associated with pulsations of the internal jugular vein; and
process at least one of the series of images and the inclination angle to determine a vertical height value corresponding to the highest subregion, thereby determining a JVP height.

In some example implementations of the apparatus, the processing hardware is configured such that the vertical height values are based at least on the inclination angle of the patient during collection of the series of images, and an angle of a sensing device determined relative to a longitudinal axis of the neck of the patient.

In some example implementations of the apparatus, the processing hardware is further configured to:
detect a gross motion in each image of the series of images, the gross motion corresponding to motion of the patient during collection of the series of images; and
exclude a portion of the series of images having gross motion greater than a gross motion threshold when generating the time-dependent signal for each subregion.

In some example implementations of the apparatus, the imaging apparatus is configured such that waveform segments for each subregion are representative of an intensity of electromagnetic radiation reflected from portions of the neck underlying the respective subregion.

In some example implementations of the apparatus, the imaging apparatus is configured such that intensity of the electromagnetic radiation is based on surface motion of the respective portion of the neck of the patient.

The imaging apparatus may be configured such that intensity of the electromagnetic radiation is based on perfusion of the neck of the patient.

The imaging assembly may include a cross-polarizer configured to facilitate detection of subsurface tissue perfusion and suppress detection of surface motion.

The imaging assembly may be absent of a cross-polarizer and is configured to facilitate detection of surface motion of the respective portion of the neck of the patient.

In some example implementations of the apparatus, the processing hardware is configured such that, for at least one subregion, the indication comprises a heat map characterizing the waveform segments associated with the subregion.

In some example implementations of the apparatus, the processor is further configured such that the indication comprising the heat map further comprises a representative waveform characterizing the waveform segments within the subregion.

In some example implementations of the apparatus, the synchronizing data includes electrocardiogram (ECG) data or photoplethysmography (PPG) data. The synchronizing data may include ECG data and the plurality of cycles include plurality of cardiac cycles, and the processor may be configured to temporally normalize at least a portion of the plurality of cardiac cycle to account for beat-to-beat variation. The synchronizing data may include PPG data, and the processor may be configured to subtract a delay to the PPG data to associate the PPG data with ventricular contraction timing.

In some example implementations of the apparatus, the processing hardware is configured such that for at least one subregion, the indication comprises a representative waveform shape characterizing the waveform segments associated with the subregion.

In some example implementations, the apparatus further comprises a sensor configured to detect the inclination angle, the sensor being operatively coupled to the processing hardware.

In another aspect, there is provided a method, comprising:
providing a sensing device comprising:
   a positioning element configured to fit within a suprasternal notch of a patient and facilitate alignment of the sensing device with the patient; and
   an imaging assembly rigidly coupled to and spaced from the positioning element, the imaging assembly configured to capture a series of images of at least a portion of a neck of the patient when the positioning element is contacted with the suprasternal notch and aligned with the patient;
receiving the series of images of the neck of the patient obtained over a period of time;
receiving synchronizing data of the patient over the period of time, the synchronizing data spanning a plurality of cardiac cycles;
defining, within the series of images, a region of interest comprising at least a portion of the neck of the patient;
for each subregion of a plurality of subregions defined within the region of interest:
   generating a time-dependent signal associated with pixel intensities within the subregion in the series of images;
   employing features within the synchronizing data to identify, within the time-dependent signal, a plurality of waveform segments, each waveform segment corresponding to a different cycle; and
determining, based on user input or sensor data, an inclination angle of the patient;
processing at least one of the series of images and the inclination angle to determine vertical height values corresponding to a plurality of locations within the region of interest;
generating a composite image comprising:
   an image comprising the region of interest;
   for at least one subregion:
      an indication characterizing at least one attribute of the waveform segments associated with the subregion;
      the indication being rendered in spatial association with the subregion; and
      the vertical height values, displayed within the region of interest, thereby facilitating a determination of the vertical height corresponding to each indication.

In another aspect, there is provided an apparatus, comprising:
a sensing device comprising:
   a positioning element configured to fit within a suprasternal notch of a patient and facilitate alignment of the sensing device with the patient; and
   an imaging assembly rigidly coupled to and spaced from the positioning element, the imaging assembly configured to capture a series of images of at least a portion of a neck of the patient when the positioning element is contacted with the suprasternal notch and aligned with the patient; processing hardware operatively coupled to the sensing device, the processing hardware comprising a memory and a processor, the memory comprising instructions executable by the processor for performing operations comprising:
   receive the series of images of the neck of the patient obtained over a period of time;
   receive synchronizing data of the patient over the period of time, the synchronizing data spanning a plurality of cardiac cycles;
   define, within the series of images, a region of interest comprising at least a portion of the neck of the patient;
   for each subregion of a plurality of subregions defined within the region of interest:
      generate a time-dependent signal associated with pixel intensities within the subregion in the series of images;
      employ features within the synchronizing data to identify, within the time-dependent signal, a plurality of waveform segments, each waveform segment corresponding to a different cycle; and
   process the waveform segments associated with each subregion to identify one or subregions associated with pulsations of an internal jugular vein;
   determine, based on user input or sensor data, an inclination angle of the patient;
   process at least one of the series of images and the inclination angle to determine a vertical height value corresponding to a convergence of sub-regions associated with pulsations of the internal jugular vein to the highest point along the neck, thereby determining a JVP height.

In some example implementations of the apparatus, the processing hardware is further configured to:
process at least one of the series of images to identify a fiducial marker residing proximal to an ear lobe of the patient; and
employ the location of the fiducial marker, and a pre-determined reference location, to construct a reference line associated with a location of an internal jugular vein; and
employ the reference line when identifying the one or more subregions associated with pulsations of the internal jugular vein.

In another aspect, there is provided an apparatus, comprising:
a sensing device comprising:
   a positioning element configured to fit within a suprasternal notch of a patient and facilitate alignment of the sensing device with the patient; and
   an imaging assembly rigidly coupled to and spaced from the positioning element, the imaging assembly configured to capture a series of images of at least a portion of a neck of the patient when the positioning element is contacted with the suprasternal notch and aligned with the patient;
processing hardware operatively coupled to the sensing device, the processing hardware comprising a memory and a processor, the memory comprising instructions executable by the processor for performing operations comprising:
   receive the series of images of the neck of the patient obtained over a period of time;
   receive synchronizing data of the patient over the period of time, the synchronizing data spanning a plurality of cardiac cycles;
   define, within the series of images, a region of interest comprising at least a portion of the neck of the patient;
   for each subregion of a plurality of subregions defined within the region of interest:
      generate a time-dependent signal associated with pixel intensities within the subregion in the series of images;
      employ features within the synchronizing data to identify, within the time-dependent signal, a plurality of waveform segments, each waveform segment corresponding to a different cycle; and
   process the waveform segments associated with each subregion to identify one or subregions associated with pulsations of an internal jugular vein;
   determine, based on user input or sensor data, an inclination angle of the patient;
   process at least one of the series of images and the inclination angle to determine vertical height values respectively corresponding to a plurality of locations within the region of interest;
   generate a composite image comprising:
      an image comprising the region of interest; and
      an indication of the subregions associated with pulsations of the internal jugular vein;
      the vertical height values, displayed within the region of interest, thereby facilitating a determination of a height of convergence of sub-regions associated with pulsations of the internal jugular vein to a highest point, relative to a fixed anatomic reference of the patient.

In some example implementations of the apparatus, the processing hardware is configured such that processing the waveform segments associated with each subregion to determine one or subregions associated with pulsations of the internal jugular vein comprises:
for each subregion, processing the waveform segments to obtain a representative waveform segment; and
processing the representative waveforms to determine one or more representative waveforms associated with pulsations of the internal jugular vein, thereby identifying one or more associated subregions associated with pulsations of the internal jugular vein.

In some example implementations of the apparatus, the processing hardware is further configured to:
process at least one of the series of images to identify a fiducial marker residing proximal to an ear lobe of the patient; and
employ the location of the fiducial marker, and a pre-determined reference location, to construct a reference line associated with a location of an internal jugular vein; and
employ the reference line when identifying the one or more subregions associated with pulsations of the internal jugular vein.

In another aspect, there is provided an apparatus, comprising:
a memory;
a processor operatively coupled to the memory and being configured to:
   receive a series of images of a target portion of a patient obtained over a period of time;
   receive synchronizing data of the patient over the period of time, the synchronizing data spanning a plurality of physiological cycles;
   define, within the series of images, a region of interest comprising at least a portion of the neck of the patient;
   for each subregion of a plurality of subregions defined within the region of interest:
      generate a time-dependent signal associated with pixel intensities within the subregion in the series of images;
      employ features within the synchronizing data to identify, within the time-dependent signal, a plurality of waveform segments, each waveform segment corresponding to a different cycle; and
   generate a composite image including:
      an image comprising the region of interest; and
      for at least one subregion:
         an indication of a representative waveform shape characterizing the waveform segments associated with the subregion;
         the indication being rendered in spatial association with the subregion.

In some example implementations of the apparatus, the processor is configured such that for at least one subregion, the indication is generated by:
processing the waveform segments corresponding to the subregion to obtain a representative waveform segment for the subregion; and
employing the representative waveform segment to generate the indication of the representative waveform shape.

In another aspect, there is provided an apparatus, comprising:
a memory;
a processor operatively coupled to the memory and being configured to:
   receive a series of images of a neck of a patient obtained over a period of time spanning a plurality of respiratory cycles;
   receive synchronizing data of the patient over the period of time, the synchronizing data spanning a plurality of respiratory cycles;
   define, within the series of images, a region of interest comprising at least a portion of the neck of the patient;
   for each subregion of a plurality of subregions defined within the region of interest:
      generate a time-dependent signal associated with changes in pixel intensities due to motion of a surface of the target portion within the subregion in the series of images;
      filter the time-dependent signal to isolate motion associated with respiration, thereby obtaining a time-dependent respiratory signal;
      employ features within the synchronizing data to identify, within the time-dependent respiratory signal, a plurality of waveform segments, each waveform segment corresponding to a different respiratory cycle; and
      employ features within the time-dependent respiratory signal to identify a plurality of waveform segments, each waveform segment corresponding to a different respiratory cycle; and
   generate a composite image including:
      an image comprising the region of interest; and
      for at least one subregion:
         an indication characterizing at least one attribute characterizing the waveform segments associated with the subregion;
         the indication being rendered in spatial association with the subregion.

In another aspect, there is provided an apparatus, comprising:
a memory;
a processor operatively coupled to the memory and being configured to:
   receive a series of images of a target portion of a patient obtained over a period of time;
   receive synchronizing data of the patient over the period of time, the synchronizing data spanning a plurality of physiological cycles;
   define, within the series of images, a region of interest comprising at least a portion of the neck of the patient;
   for each subregion of a plurality of subregions defined within the region of interest:
      generate a time-dependent signal associated with changes in pixel intensities due to motion of a surface of the target portion within the subregion in the series of images;
      employ features within the synchronizing data to identify, within the time-dependent signal, a plurality of waveform segments, each waveform segment corresponding to a different cycle; and
   generate a composite image including:
      an image comprising the region of interest; and
      for at least one subregion:
         an indication characterizing at least one attribute of the waveform segments associated with the subregion;
         the indication being rendered in spatial association with the subregion.

In another aspect, there is provided an apparatus, comprising:
a memory;
a processor operatively coupled to the memory and being configured to:
   receive a series of images of a neck of a patient obtained over a period of time;
   receive synchronizing data of the patient over the period of time, the synchronizing data spanning a plurality of physiological cycles;
   define, within the series of images, a region of interest comprising at least a portion of the neck of the patient;
   for each subregion of a plurality of subregions defined within the region of interest:
      generate a time-dependent signal associated with pixel intensities within the subregion in the series of images;
      employ features within the synchronizing data to identify, within the time-dependent signal, a plurality of waveform segments, each waveform segment corresponding to a different cycle; and
generate a composite image comprising:
   for at least one subregion:
      an indication characterizing at least one attribute of the waveform segments associated with the subregion;
      the indication being rendered in spatial association with the subregion.

In another aspect, there is provided an apparatus, comprising:
a memory;
a processor operatively coupled to the memory and being configured to:
   receive a series of images of a target portion of the patient obtained over a period of time;
   receive synchronizing data of the patient over the period of time, the synchronizing data spanning a plurality of physiological cycles;
   define, within the series of images, a region of interest comprising at least a portion of the neck of the patient;
   for each subregion of a plurality of subregions defined within the region of interest:
      generate a time-dependent signal associated with pixel intensities within the subregion in the series of images;
      employ features within the synchronizing data to identify, within the time-dependent signal, a plurality of waveform segments, each waveform segment corresponding to a different cycle;
   process the waveform segments associated with each subregion to determine at least one set of subregions characterized by similar waveform segments, each set of subregions defining a respective subregion cluster; and
   generate a composite image including:
      an image comprising the region of interest; and
      for at least one subregion cluster:
         an indication of a location and spatial extent of the subregion cluster.

In another aspect, there is provided an apparatus, comprising:
a memory;
a processor operatively coupled to the memory and being configured to:
   receive a series of images of a target portion of a patient obtained over a period of time;
   receive synchronizing data of the patient over the period of time, the synchronizing data spanning a plurality of physiological cycles;
   define, within the series of images, a region of interest comprising at least a portion of the neck of the patient;
   for each subregion of a plurality of subregions defined within the region of interest:
      generate a time-dependent signal associated with pixel intensities within the subregion in the series of images;
      employ features within the synchronizing data to identify, within the time-dependent signal, a plurality of waveform segments, each waveform segment corresponding to a different cycle; and
   generate a composite image including:
      an image comprising the region of interest; and
      for at least one subregion:
         an indication comprising a heat map generated based on the waveform segments associated with the subregion;
         the indication being rendered in spatial association with the subregion.

In some example implementations of the apparatus, the processor is further configured such that the indication further comprises a representative waveform characterizing the waveform segments within the subregion.

In another aspect, there is provided an apparatus, comprising:
a memory;
a processor operatively coupled to the memory and being configured to:
   receive a series of images of a target portion of a patient obtained over a period of time;
   receive synchronizing data of the patient over the period of time, the synchronizing data spanning a plurality of physiological cycles;
   define, within the series of images, a region of interest comprising at least a portion of the neck of the patient;
   for each subregion of a plurality of subregions defined within the region of interest:
      generate a time-dependent signal associated with pixel intensities within the subregion in the series of images;
      employ features within the synchronizing data to identify, within the time-dependent signal, a plurality of waveform segments, each waveform segment corresponding to a different cycle; and
   generate a composite image including:
      an image comprising the region of interest; and
      for at least one subregion:
         an indication characterizing at least one attribute of the waveform segments associated with the subregion;
         the indication being rendered in spatial association with the subregion.

### BRIEF DESCRIPTION OF THE DRAWINGS

For a better understanding of the present technology, as well as other aspects and further features thereof, reference is made to the following description, which is to be used in conjunction with the accompanying drawings, where:

FIG. 1A shows an illustration of a patient lying on a bed at an angle and illustrates the vertical distance relative to the sternal angle on the IJV of the patient that can be used to determine the JVP height of the patient.

FIG. 1B shows plots of electrocardiogram (ECG) data and JVP data of a patient, and corresponding imaging photoplethysmography (PPG) data obtained from the patient.

FIG. 2 is a schematic block diagram of a system for generating visual information of a patient's physiological parameter such as the patient's JVP, according to an embodiment.

FIG. 3A is a schematic block diagram of a sensing system for measuring a patient's JVP, according to an embodiment.

FIGS. 3B and 3C illustrate an example implementation of a system that facilitates measurement of a patient's JVP height.

FIG. 3D is an image acquired from an example sensing device in the absence of positioning the sensing device on a patient, illustrating how the portion of the sensing device that is captured by the image is static and will not change location when the device is used on a patient.

FIG. 3E shows an outline of the patient prior to annotation, showing, within the outline, the properly positioned sensing device.

FIG. 4 is a schematic block diagram of a control unit that may be included in the system of FIG. 2, according to an embodiment.

FIGS. 5A and 5B depict mean intensity with each pixel within a node of a region of interest being assigned a weight of 1.

FIGS. 6A and 6B depict weighted mean intensity with each pixel within a node of a region of interest being assigned different weights.

FIG. 7A schematically depicts the placement of nodes on a neck of a patient, according to an embodiment.

FIGS. 7B and 7C illustrate examples of a fiducial marker that may be positioned on the patient to facilitate the positioning of the region of interest in the images acquired by the imaging assembly, and to facilitate identification of JV pulsations associated with the IJ (EJ) vein.

FIG. 7D shows the annotation of an image with a region of interest and a reference line, the reference line having been generated, in part, based on the location of the fiducial marker applied to the patient.

FIG. 8 shows a plot of ECG data obtained from a patient during collection of a series of images of a neck of the patient that includes a plurality of cardiac cycles and time-varying node signals obtained from the series of images temporally synchronized with the ECG data, according to an embodiment.

FIG. 9A is a composite image including an image of the user's neck and visual elements representative of spatio-temporal vascular pulsations overlaid on the image, in which the visual elements are provided for a plurality of nodes or regions, each of which displays averaged visual indication of the patient's pulsatile variations for at least a portion of a cardiac cycle obtained from a corresponding region of the patient's neck, according to an embodiment.

FIGS. 9B and 9C are composite images including spatially clustered waveform indicators, according to embodiments.

FIGS. 10A and 10D show composite images that include an image of the user's neck and visual elements representative of vascular pulsations overlaid on the image, in which the visual elements are provided for a plurality of nodes or regions, each of which displays representative visual indications of the patient's pulsatile variations for at least a portion of a cardiac cycle obtained from a corresponding region of the patient's neck, according to an embodiment.

FIGS. 10B and 10E show heat maps of a portion of the plurality of nodes of FIG. 10A, according to an embodiment; FIGS. 10C and 10F show an enlarged view of a portion of the heat maps of FIG. 10B indicated by the arrow A in FIG. 10B.

FIG. 11A is a composite image including an image of the user's neck and visual elements representative of vascular pulsations overlaid on the image, in which the visual elements are provided for a plurality of nodes or regions, each of which displays representative visual indication of the patient's pulsatile variations for at least a portion of a cardiac cycle obtained from a corresponding region of the patient's neck, according to an embodiment.

FIG. 11B shows heat maps of a portion of the plurality of spatial segments of FIG. 11A, according to an embodiment.

FIG. 12 is a composite image including an image of the user's neck and visual elements indicative of vascular pulsations overlaid on the image, in which the visual elements are provided for a plurality of nodes or regions, each of which displays averaged visual indication of the patient's pulsatile variations for at least a portion of a cardiac cycle obtained from a corresponding portion of the patient's neck, and having height markers indicative of a vertical height of different points on the patient's neck, according to an embodiment.

FIG. 13 shows an optical image of a neck of a patient having a sensing device of a sensing system disposed on the patient's chest such that at least a portion of the sensing device is disposed in a sternal notch of the patient, and an orientation of the sensing device relative to a neck orientation of the neck of the patient is depicted, according to embodiments.

FIGS. 14A-14C show an image of a patient's neck with height markers overlaid on the image, according to various embodiments.

FIG. 15A shows a cylindrical model of a neck of a patient that may be used to determine height information, according to an embodiment.

FIG. 15B shows a conical shaped model of a neck of a patient that may be used to determine height information, according to an embodiment.

FIG. 15C shows the cylindrical model of FIG. 15A with a vertical height projected onto a surface of the cylinder relative to a sternal notch of the patient where a sensing device may be disposed, according to an embodiment.

FIG. 15D shows an optical image of a neck of a patient with a cylindrical model of the neck of the patient including vertical height markers overlaid on portion the neck model, according to an embodiment.

FIG. 16A shows side views of a portion of a human skeleton showing the sternal notch and portion of the neck coupled to the sternal notch.

FIG. 16B shows an orientation angle of the sensing device relative to true horizontal, which can be used to determine an inclination angle of the patient to accurately estimate vertical height markers for superimposing on the composite image, according to an embodiment.

FIG. 17A shows plots of respiratory motion of the patient obtained from imaging a region of interest of a neck of a patient over a plurality of respiratory cycles, according to an embodiment.

FIGS. 17B and 17C shows a composite image that includes an image of a neck of a patient and respiratory motion information overlaid over the selected image, with the region of interest including an average phase and amplitude of respiratory motion of the patient corresponding to a plurality of spatial nodes or regions of the neck of the patient, according to an embodiment.

FIG. 17D illustrates features and phases of a respiratory cycle.

FIGS. 18A-18B show a schematic flow chart of a method for visually displaying one or more physiological parameters associated with a patient, which can be determined from a series of images of the neck of a patient, according to an embodiment.

### DETAILED DESCRIPTION

Systems, devices, and methods described herein enable generation of images and other visual elements for assisting a clinician in evaluating one or more physiological conditions of a patient, including, for example, cardio-respiratory conditions of a patient. In some embodiments, systems, devices, and methods described herein can generate a composite image that includes a visual image of a target portion of a user's body, such as a neck, which is overlaid with visual elements indicative of a patient's physiological information (e.g., cyclical physiological information such as that associated with cardiac cycles or respiration cycles). In some embodiments, the visual elements can show temporally segmented and averaged values of time-varying pulsation information obtained using electromagnetic signals measured by a sensing system. The target portion or region of interest can be divided or segmented into a plurality of nodes or areas, with a visual element displayed overlaying each node or area that represents or is associated with the information measured for that node or area. The images and visual elements provided by systems, devices, and methods described herein can enable a medical professional or caregiver to observe both the spatial and temporal relationship of underlying signals with one or more static images. In some embodiments, heat maps or other visual elements can also be presented to allow a physician to evaluate the quality or reliability of the representative signal information.

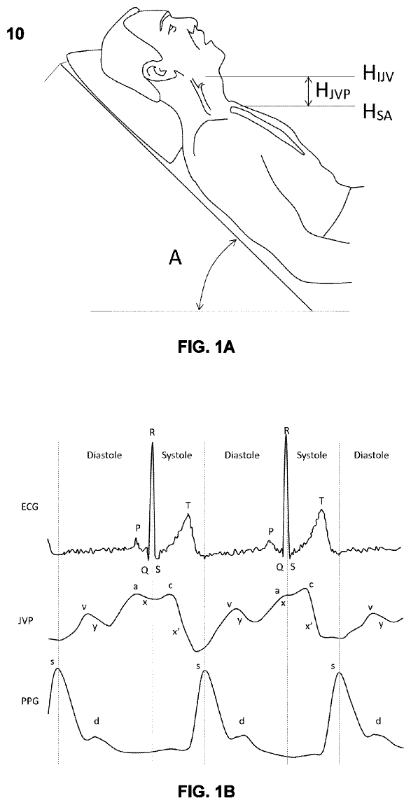

In some embodiments, sensing devices described herein for collecting patient information can use imaging, for example, imaging photoplethysmography (iPPG) to collect images or videos of a patient's neck and determine the JVP height of the patient based on electromagnetic radiation reflected from the neck of the patient. The sensing devices can capture time-varying pulsatile information of a patient and, in particular, capture information associated with venous and/or arterial pulsations. Arterial and venous pulsations are generally out of phase. There are some notable differences in the setting of pathology such as atrial fibrillation, tricuspid regurgitation, etc. Arterial pulsations occur after the R-wave whereas the venous pulsation (a-c wave) generally straddle the R-wave. Arterial pulsations generally have a sharp upslope while venous pulsation have a more gradual upslope and steeper downslope. For example, FIG. 1B shows ECG data and corresponding JVP and arterial PPG data obtained from a patient. The JVP waveform is generally formed by three ascents (a, c, and v waves) and three descents (x, x′, and y), which respectively represent the different events of the cardiac cycle in terms of pressure variations in the jugular vein. The PPG waveform captures arterial pressure variations, where s indicates the systolic peak and d indicates the diastolic peak. Both JVP and arterial PPG data vary in time due to events during each cardiac cycle.

While monitoring the arterial PPG signal may enable assessment of cardiac pump efficiency and blood delivery function, it may not capture instances of central venous pressure abnormalities associated with the right side of the heart. Systems, devices, and methods described herein can address this limitation by using iPPG to capture data of cyclic vascular pulsatile motion, including motion that is associated with the internal jugular vein. One of the main challenges with JVP height determination based on iPPG is extraneous motion, for example, caused by motion of the patient and/or neck of the patient unrelated to cyclic vascular pulsatile motion, and differentiating this unwanted motion from cyclic vascular pulsatile motion due to pressure waves propagated into the venous vasculature during cardiac contraction. Motion observed in an image of a patient can be broken down into gross motion, i.e., motion of the entire body or portion of the body of the patient (e.g., patient moving head and neck during imaging) and regional motion, i.e., motion within specific regions of the target portion of the patient's body (e.g., motion in portions of neck due to sporadic muscular contraction, swallowing, speaking, etc.). Gross motions or regional motions unrelated to the cyclic vascular pulsatile motion of the patient are undesirable as they contribute to noise and degrade reliability of the iPPG signals to identify vascular pulsations obtained from a neck of the patient.

Embodiments of the systems and methods described herein for visualizing physiological parameters, and particularly, pulsatile variations and/or any other cardiorespiratory parameters of a patient obtained using electromagnetic imaging may provide one or more benefits including, for example: (1) reducing noise by identifying repetitive signals in a series of images to isolate repetitive pulsatile motion signals associated with the JVP, other vessels or respiration from random motion; (2) reducing noise by identifying and removing one or images from a series of images that have motion beyond a motion threshold, and/or using motion stabilization techniques to reduce motion during image capture; (3) facilitating identification of the JVP from a patient by a medical professional by displaying a static image (e.g., composite image) including spatially segmented and representative recurring temporal waveforms serving as visual indicators of pulsatile motion or another cardiorespiratory parameter of the patient overlaid on an image of a neck of a patient, along with height indicators of the neck; (4) enabling quantification of one or more cardiorespiratory parameters by displaying representations of cyclical signals amplitude and/or phase of the signal (e.g., electromagnetic signal) corresponding the patient's physiological parameter on a static image; and/or (6) automatically determining and/or displaying physiological parameters such as, for example, an ECG, heart rate, JVP height, respiration rate, blood pressure, blood oxygen level, and/or any other physiological parameter.

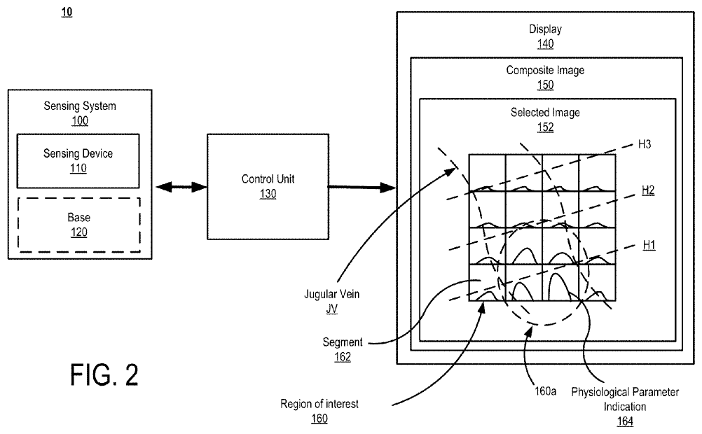

FIG. 2 is a schematic block diagram of a system 10 including a sensing system 100 for measuring a patient's vascular pulsatile motion and/or other physiological parameters or conditions, and a control unit 130 configured to generate a visual representation of the patient's vascular pulsatile motion or other physiological parameter on a display 140, according to an embodiment. In some embodiments, the control unit 130 may be separate from the sensing system 100, as shown in FIG. 2, for example, include a laptop, a remote server, or a cloud computing system. In some embodiments, the control unit 130 may be integrated in the sensing system 100, for example, integrated in a base 120 of the sensing system 100. In some embodiments, the display 140 may also be integrated in the sensing system 100. In some embodiments, the display 140 may be integrated with control unit 130 in a system that is separate from the sensing system 100.

The system 100 or any other system or sensing system described herein may be configured to measure or monitor vascular pulsatile or cyclic motion (e.g., IJV or EJV pulsation), carotid (arterial) pulsation, respiratory effort (normal and/or distressed), or may be use for monitoring patients suffering from CHF, COPD, asthma, dialysis (both peritoneal and hemodialysis), pericardial tamponade, conditions resulting in elevated intracardiac pressures, scenarios in which excess circulating blood volume is an issue (e.g., sceptic shock after volume resuscitation with IV fluid), or any other disease or condition. All such measurements, determinations, or diagnosis are contemplated and should be considered to be within the scope of the present disclosure.

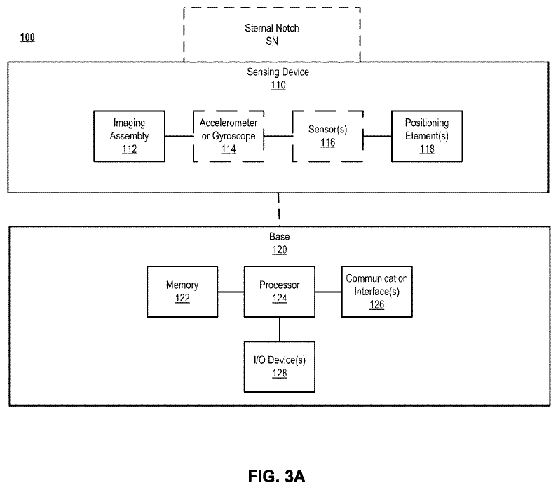

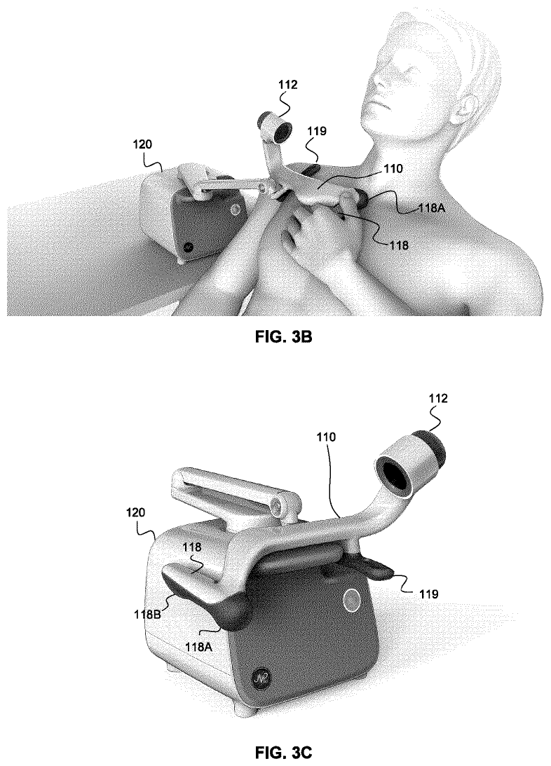

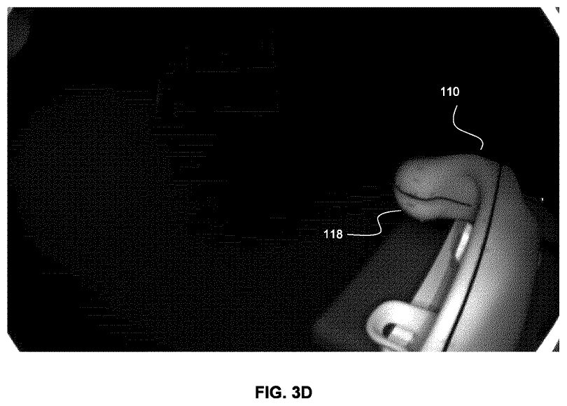

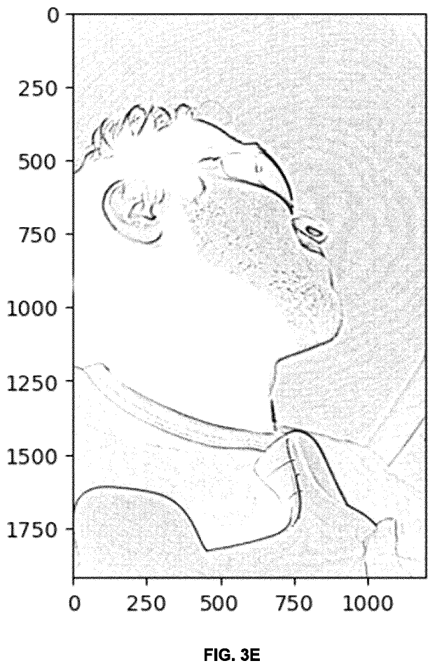

The sensing system 100 may include a sensing device 110 and, optionally, a base 120. For example, FIG. 3 is a schematic block diagram of the sensing system 100 for measuring a patient's JVP and/or other physiological parameters or conditions, according to a particular embodiment. It should be appreciated that the sensing system 100 shown in FIG. 3 is only an example, and any other system capable of capturing information regarding one or more physiological parameters or conditions of a patient may be used as the sensing system 100 in system 10. Various examples of sensing systems that can be used as the sensing system 100 in the system 10 of FIG. 2 are described in detail in PCT Application No. PCT/CA2022/051177 (the “'177 application”), filed Aug. 2, 2022, and entitled “Apparatuses, Systems, and Methods for Capturing a Video of a Human Patient Suitable for Monitoring a Cardiac, Respiratory or Cardiorespiratory Condition,” the entire disclosure of which is incorporated herein by reference.

Referring to FIG. 3A, an example system 100 is shown that includes a sensing device 110 that includes an imaging assembly 112 and a positioning element(s) 118, and optionally, an accelerometer or gyroscope 114 and/or one or more additional sensor(s) 116. The system 100 can also include the base 120 that may include a memory 122, a processor 124, a communication interface(s) 126, and an input/output (I/O) devices(s) 128. While shown as being separate from the sensing device 110, the components of the base 120 may be integrated within the sensing device 110 such that a separate base 120 coupled to the sensing device 110 is excluded. In some embodiments, the sensing device 110 is configured to capture a series of images of a neck of a patient or electromagnetic radiation reflected by a neck of a patient, and allow determination of the JVP therefrom. The sensing device 110 may be operatively coupled to the base 120 and configured to capture one or more images of the neck or reflected electromagnetic radiation based on an activation signal received from the base. The sensing device 110 can also be configured to transmit captured images and/or signals to the base 120.

The sensing device 110 may include a housing (not shown) within which components of the sensing device, for example, the imaging assembly 112, the accelerometer or gyroscope 114, the sensor(s) and/or the positioning element 118 may be disposed, coupled to, integrated with, or monolithically formed therewith. The imaging assembly 112 is configured to capture a single image, a set or series of images, and/or a video of a portion of a body of the patient (e.g., a neck of a patient). For example, the imaging assembly 112 may be used to capture an image, a set or series of images, or a video of a neck of a patient in an area of the neck where the distention of the IJV is visible to determine the JVP of a patient. In various embodiments, the imaging assembly 112 may include one or more imagers such as, for example, a charged coupled device (CCD) camera configured to capture the image(s) and/or video(s) of the portion of the body of the patient.

In some embodiments, the imaging assembly 112 may also include one or more lenses (e.g., concave and/or convex lenses), and/or one or more filters (e.g., optical filters, software filters such as high pass filter(s), low pass filter(s), bandpass filter(s), etc.) to facilitate capture of images (e.g., at different wavelengths), to reduce noise, and/or increase image focus. The imaging assembly 112 may be positionable with respect to the neck of the patient to be able to capture image(s) and/or video of the patient so as to be able to capture distention of the patient's IJV and thereby, the patient's JVP. The imaging assembly 112 generates a signal indicative of the captured images and may communicate the signal to the base 120 (e.g., to the processor 124 included in the base 120).

In some embodiments, the imaging assembly 112 may also include a light source or an illumination mechanism (e.g., an electromagnetic radiation source such as one or more light emitting diodes (LEDs)) to illuminate the portion of the body (e.g., the neck) of the patient. The light source can be configured to generate sufficient illumination for a camera of the imaging assembly 112 to image the patient's body. In some embodiments, the illumination mechanism can be configured to generate light for capturing one or more images or a video. For example, the illumination mechanism can be configured to illuminate an area for optical imaging.

In some embodiments, the illumination mechanism may be configured to generate a reference element, for example, to provide a spatial reference having known characteristics (e.g., size, shape, distance, etc.) that may be employed during calculation of the JVP of the patient (e.g., via one or more mathematical calculations). For example, the illumination mechanism may be configured to project electromagnetic radiation (e.g., visible light, infrared or ultraviolet (UV) light) in the form of a reference image or reference element, for example, onto the neck of the patient that is within the field of view of the imager included in the imaging assembly 112. In some embodiments, the reference image may include one or more lines (e.g., three parallel lines, or any other suitable marking) of projected light onto an appropriate area on the neck of the patient. In some embodiments, the reference image may have markings that can facilitate mathematical calculations to be performed. For example, in some embodiments, the markings on the reference element may be one or more scales of measurement (e.g., centimeters). In some embodiments the markings may include numbers. In some embodiments, the markings may be or may include but are not limited to, different patterns, colors, or other differentiations in structured electromagnetic radiation.

In other embodiments, the reference element may be a separate physical element that may be positioned on the neck of the patient by the patient, a caregiver, or a medical practitioner, before imaging the neck of the patient by the imaging assembly 112. In operation, the sensing device 110 may be disposed on the body of the patient such that the imaging assembly 112 is positioned in a desired orientation with respect to the neck of the patient to allow capturing of video of the neck of the patient in an area where the distention of the jugular vein is visible (e.g., by positioning at least a portion of the positioning element(s) 118 in a sternal notch SN of the patient). In its correct position, the reference element is positioned with respect to the neck of the patient such that (i) the reference element is also within the frame of the electronic imager, (ii) can be captured on video being recorded by the imaging assembly 112, and (iii) mathematical calculations can be made from a captured video to calculate the JVP of the patient, and example methods of calculating the JVP are described in detail below.

In some embodiments, the imaging assembly 112 may include an illumination mechanism implemented as an electromagnetic radiation source providing electromagnetic radiation in at least the near infrared (NIR) spectrum, and a filter allowing for the selective passage of electromagnetic radiation in the NIR spectrum to the imager included in the imaging assembly 112. In some such embodiment, the apparatus being so structured assists in reducing issues associated with ambient lighting. For example, artificial ambient lighting can introduce noise into the signals due to flickering. Having a dedicated light source in the NIR spectrum, and a filter only allowing passage of electromagnetic radiation in the NIR spectrum may substantially reduce noise due to flickering or other optical lighting noise. NIR imaging can also reduce the impact of skin tone on image processing. In some embodiments, the imaging assembly 112 may include a cross-polarization filter (e.g., disposed over an illumination source included in the imaging assembly 112 and/or a lens of one or more imagers of the imaging assembly) configured to reduce specular reflection.

Image PPG primarily relies on surface reflection, where it detects variations in the intensity of electromagnetic energy that is reflected from the skin's surface. These surface reflections are influenced by the presence of blood vessels within the subcutaneous tissue, and as blood circulates through these vessels, it modulates the absorption and scattering of light. Some research applications have delved into detecting subsurface reflection, which involves capturing light that penetrates deeper beneath the skin's surface. While subsurface methods are believed to offer greater accuracy and robustness, they often require specialized equipment.

However, there are several challenges associated with employing surface reflection-based image PPG techniques to determine jugular venous pressure height. Motion from the subject and regional tissues can introduce noise, making it difficult to extract a clean PPG signal. Moreover, the internal jugular (IJ) vein is relatively deep beneath the skin's surface, limiting its interaction with incident energy. Maintaining a consistent signal in the presence of noise and addressing signal drift over time compounds the complexity of surface reflection image PPG. Consequently, the primary image PPG signal from surface reflection is generated from tissue motion with a smaller component due to the interaction with superficial vessels.

One innovative approach described in this text addresses the challenging task of distinguishing between the motion associated with respiration and the underlying vessels from that of skeletal muscular contraction. The solution leverages the fact that vessel pulsations cause surface tissue motion that exhibit pseudo-periodic characteristics, have distinct timing relative to ventricular contraction, and possess anatomic and physiologic features specific to the carotid, IJ vein, and jugular venous pressure (JVP) that enable the isolation of these signals. Additionally, respiration generates superficial tissue motion on the neck surface which is also pseudo-periodic in nature and occurs with distinct timing relative to that start & end of inspiration and expiration. It is also recognized that this approach can be applied to subsurface signals in a similar fashion as described.

In some embodiments, the imaging assembly 112 may include a plurality of imagers. For example, the imaging assembly 112 may include a first electronic imager, and a second electronic imager fixed in position with respect to the first electronic imager. Having two imagers that are a fixed and known distance apart may enable stereo vision or depth sensing through a calculation of a disparity or difference map. This can enable generation of a 3D model of the structure (e.g., the neck) of the patient being imaged. In some embodiments, the first electronic imager may be an NIR spectrum imager, and the second electronic imager may be a visible light spectrum imager. In some such embodiments, the NIR spectrum imager captures images in greyscale, whereas the visible light spectrum imager captures images in color. Thus, the two imagers provide different information that can be helpful for image processing, segmentation and feature identification. The color information in combination with the NIR information can assist with feature identification and/or segmenting different objects from one another (e.g., clothing from skin, or the outline of the body from the backgrounds).

In some embodiments, where the JVP of the patient is being monitored, the reference element that may be generated by an illumination mechanism (e.g., a visible or NIR electromagnetic radiation source) included in the imaging assembly 112 or included in another part of the sensing device (e.g., in a positioning element or locator object), may have predetermined dimensions that render a height of the column of blood in a jugular vein of the patient with respect to at least one of the patient's sternal angle and/or another grossly immobile anatomic feature of the patient's torso determinable by the processor 124, for example, from the captured image(s) or video of the neck or portion of the neck of the patient and a locator object (e.g., positioning element 118). In some such embodiments, another grossly immobile anatomic feature of the patient's torso is one of a patient's clavicular head, suprasternal notch, and sternum.

In some embodiments, the height of the column of blood in the jugular vein of the patient with respect to at least one of the patient's sternal angle and another grossly immobile anatomic feature of the patient's upper body is determinable by the processor 124 from the captured electronic video without having reference to a different image of the neck of the patient and/or without requiring the imager having been fixed in a precise position with respect to the neck of the patient at the time that the electronic video was captured. For example, the reference element being of a known size can facilitate the measurement of the JVP. For example, the known size of the reference can enable the conversion between pixels (e.g., of the video captured by the imager) and a physical distance. In some embodiments, a clinician may be able to review a video, static image(s), or other visualization elements generated based on information captured by the imaging assembly 112 and determine the location of the top of the pulsation in the IJV. Alternatively or additionally, the processor 124 via processing the video data can be configured to identify the location of the top of the pulsation of the IJV. The distance (e.g., vertical distance) can be calculated using the number of pixels and the angle of the patient (e.g., as measured by the accelerometer or gyroscope 114).

The accelerometer or gyroscope 114 may include, for example, a microelectromechanical (MEMS) accelerometer, a piezoelectric accelerometer, a piezoresistive accelerometer, a capacitive accelerometer, a rotating gyroscope, a vibrating gyroscope, an optical gyroscope, any other suitable accelerometer or acceleration sensing device, or a combination thereof. In some embodiment, the accelerometer or gyroscope 114 may be used to determine whether the imaging assembly 112 is moving, for example, due to the sensing device 110 (e.g., the positioning element 118 of the sensing device 110) not stably positioned on the body of the patient as described herein. In some embodiments, the processor 124 included in the base 120 may be configured to receive a signal from the accelerometer or gyroscope 112, and based on the accelerometer or gyroscope signal, activate the imaging assembly 112 based on determining that the sensing device 110 is sufficiently stably disposed on the patient's body (e.g., is stationary or is moving less than a predefined amount or rate) for the imaging assembly 112 to capture clear image(s) or video(s) of the portion of the patient's body.

In another example, the accelerometer or gyroscope 114 can also be used to determine the angle of the patient with respect to a horizontal axis. For example, when measuring a patient's JVP, the angle at which the patient's torso and neck are inclined to the horizonal may be between about 30 degrees to about 60 degrees, inclusive. Depending on the specific angle that the patient is inclined, the vertical height or distance between the sternal angle and the top of the pulsation point of the IJV may change, e.g., as described above with reference to FIG. 1A. Thus, the accelerometer or gyroscope 114 may be configured to measure and communicate the angle of inclination to the processor 124 of the base 120. In some embodiments, the processor 124 may be configured to activate the imaging assembly 112 when the patient is inclined at an angle between about 30 degrees and about 60 degrees, inclusive, and the sensing device 110 is stably disposed on the patient's body. In some embodiments, the processor 124 can use the angle data acquired by the accelerometer or gyroscope 114 in determining a vertical distance between the sternal angle and top of the pulsation point of the IJV, for example, to determine the JVP.

The sensing device 110 may also include one or more sensors 116 that may be configured to sense at least one physiological parameter of the patient. In some embodiments, at least a portion of the sensor(s) 116 may project outward from the housing of the sensing device 110, or be generally accessible through the housing so as to able to contact a portion of the body of the patient (e.g., sternum or chest of the patient, or a hand of a patient) and measure one or more physiological parameters of the patient. In some embodiments, the sensor(s) 116 may include a contact sensor, an ECG electrode, a heart rate sensor, a PPG sensor, a blood pressure sensor, a blood oxygen sensor, any other suitable sensor, or a combination thereof. In some embodiments, the sensor(s) 116 may be configured to contact the patient's skin, for example, a skin of a sternum or torso of the patient, or provided on grips defined on the housing of the sensing device 110 so as to contact skin of a hand or one or more fingers of the patient when the patient grips the sensing device to measure the patient's JVP (or any other physiological parameter described herein). In some embodiments, the sensor(s) 116 may include at least two electrodes for contacting the skin of the patient to capture an ECG signal. In some embodiments, the one or more of the electrodes may be in contact with the skin of the patient when the sensing device 110 is properly positioned on a patient for image capture (e.g., by imaging assembly 112). For example, in some embodiments it may be that one of the electrodes is on a locator object (e.g., positioning element 118) and is positioned on the locator object such that when the locator object is correctly positioned on the patient's body, the electrode is in contact with the patient's skin.

In some embodiments, the sensor(s) 116 may be used to send a signal that the sensing device 110 has been positioned on the patient's body. In some embodiments, a signal from the sensor(s) 116 in combination with the signal from the accelerometer 112 whose signal can be used to determine whether the sensing device 110 is moving at rate beyond a predetermined threshold (e.g., at rate of greater than 0.01 mm/second or about 1 mm/second, including all sub-ranges and values therebetween) and thus not yet stably positioned on the body of the patient. In some embodiments, the processor 124 may be configured to automatically start capturing video based on having received the appropriate signals from the sensor(s) 116 and/or the accelerometer or gyroscope 114, for example, that the sensing device 110 is stably positioned and that the inclination of the patient's torso and neck are within range for determining the patient's JVP.

In some embodiments, the sensor(s) 116 is synchronizable with imaging data from the imaging assembly 112, for example, by the processor 124 included in the base 120 or some other processor that is operatively coupled to the system 100 (e.g., via communication interface(s) 126). Thus, in some embodiments, the captured video and the data from the sensor(s) 116 are coordinated or synchronized. In a non-limiting example, when the sensor(s) 116 include an ECG electrode or a pulse oximeter, the imaging assembly 112 may collect data in synchroneity with the signals from the sensor(s) 116. For example, the time at which the sensor(s) 116 data is recorded and the time at which image (video) data from the imager is recorded can be known or determinable by the processor 124 in absolute and/or relative time. For example, in some embodiments, the clock of the sensor(s) 116 and/or the accelerometer 114 can be used to gate the imager (or vice versa). In some embodiments, the processor 124 generates a clock signal that gates both the imaging assembly 112 and the sensor(s) 116. In some embodiments, both the image data and the sensor data are timestamped using the same clock signal and the data is temporally aligned in a post-processing step, for example, by the processor 124 or a remote processor (e.g., a user device such as a mobile phone, a tablet, a laptop computer, or a desktop computer, or a remote server).

In some embodiments the sampling rate of the sensor(s) 116 may be a multiple of the sampling rate of the imaging assembly 112. For example, the ratio of the sensor(s) 116 data sampling rate to the imaging assembly 112 data sampling rate in some embodiments can be between about 0.5:1 to about 10:1, including all values and sub-ranges therebetween, including about 2:1. When the ratio is 2:1, the sensor(s) 116 data can be sampled at T₁, T₂, T₃, T₄, T₅, etc., whereas the imaging assembly 112 data can be sampled only at T₁, T₃, T₅, etc. (with the interval between each Tₙ and Tₙ₊₁ being a constant). In such an example, the sensor(s) 116 data may be used to assist in interpreting the imaging assembly 112 data.

In some embodiments, the sensor(s) 116 can include a microphone or other audio capture device. The audio capture device may, for example, be used to capture the sound of the patient's voice while the patient is speaking, or to capture other sounds generated by a patient (e.g., breathing, heart sounds, etc.). For example, the audio capture device can be in contact with the skin of the patient and used to capture sounds generated by the patient's body, for example, heart sounds (e.g., from the cardiac valves opening and closing); respiratory sounds (e.g., from air moving into and out of the lungs); and/or blood flow sounds from blood following in the blood vessels and/or through the valves.

The positioning element(s) 118 may be configured or structured to facilitate a correct or desired positioning of the sensing device 110 on the body (e.g., a torso or sternum) of the patient, for example, so as to position the imaging assembly 112 in the correct or desired position relative to the portion of the body of the patient (e.g., the neck of the patient) for measurement or monitoring of a condition of the patient. For example, in some embodiments, the positioning element(s) 118 can be configured to facilitate positioning of the sensing device 110 such that the portion of the body of the patient (e.g., the neck of the patient) is within the field of view of the imager of the imaging assembly 112 when the sensing device 110 is positioning on the patient as guided by the positioning element(s) 118. This may be desirable to enable the imaging assembly to perform appropriate monitoring of the JVP or any other condition or physiological parameter of the patient as described herein.

In some embodiments, the positioning element(s) 118 can include or be implemented as a locator object. For example, the positioning element(s) 118 implemented as a locator object may be structured to be positionable on the body of the patient with respect to an anatomic location on a patient. In some implementations, this may be accomplished by having at least a portion of the positioning element(s) 118 be sized and shaped to be able to register with the size and shape of a particular anatomic location of a patient's body. Examples of such anatomic locations are the patient's sternum, sternal angle, manubrium, clavicular heads, and suprasternal notch (also referred to herein as “sternal notch”).

For example, FIGS. 3A, 3B and 3C, in some example implementations, at least a portion of the positioning element(s) 118 may be configured to be disposed in the sternal notch SN of the patient. This may facilitate the patient or a medical provider to orient the imaging assembly 112 of the sensing device 110 in a desired orientation relative to the neck of the patient. In implementations where the anatomic location is the patient's sternal notch SN, such as the non-limiting example configuration shown in FIGS. 3A-3C, the positioning element(s) 118 may have a spherical portion 118A (e.g., a hemispherical shaped projection coupled to a base of the housing of the sensing device 110 or monolithically formed in the base of the housing) dimensioned to snugly fit within the sternal notch SN.

In some embodiments, the position element(s) 118 may include an interchangeable or adjustable patient-contacting element, thus allowing positioning element to be customized to the patient (e.g., to accommodate a change in size, a change in anatomic location, etc., between patients) to aid in positioning the positioning element(s) 118 on the patient's torso, and thereby correctly positioning the imaging assembly 112 relative to the portion (e.g., the neck) of the patient's body. The positioning element 118 implemented as a locator object can aid the imaging assembly (or an imager of the imaging assembly) in determining a fixed reference point (e.g., sternal angle) via processing and analysis (e.g., mathematical calculations). Such can be accomplished via imaging processing or mechanical measurement given a mechanical linkage between the imager and the locator object. As such, the positioning element(s) 118 aids in positioning the device but also enables the determination of an anatomic landmark.

In addition, or in the alterative, in some embodiments the positioning element(s) 118 has at least one adjustable patient-contacting element. Where present, an adjustable patient-contacting element also may assist in positioning the positioning element(s) 118 on the patient's torso. In other embodiments, in addition or in the alternative to the foregoing, the positioning element(s) 118 may be an interchangeable element, for example, to serve a similar purpose.

In some embodiments, the sensing device 110 may include a first positioning element configured to be positioned on a first portion of the patient's body (e.g., a spherical element or portion configured to be positioned on the suprasternal notch) and a second positioning element spaced apart from the first positioning element and configured to be positioned on a separate part of the patient's body (e.g., on a pectoral muscle or chest). Providing multiple positioning elements 118 may allow substantially more stable and correct positioning of the sensing device 110 on the torso of the patient.

FIGS. 3B and 3C illustrate an example implementation in which the sensing device 110 includes a positioning element 118 that includes a first feature 118A that is configured to uniquely fit/mate (i.e. is keyed with) the sternal notch. The positioning element is also longitudinally shaped, enabling the user/wearer to properly align the longitudinal axis of the positioning element with the body, thereby ensuring both proper positioning and proper orientation of the sensing device 110 relative to the body. Moreover, the positioning element includes a secondary positioning feature 118B that has a shape and position configured to uniquely fit/mate with the sternal angle. The secondary positioning feature 118B facilities improved positioning accuracy and also facilitates angular (orientational) alignment of the sensing device 110 relative to the body. FIGS. 3B and 3C also illustrate the example use of an additional positioning element 119 that is employed to stabilize the device relative to the body at the shoulder region.

FIGS. 3B and 3C also illustrate a configuration in which the imaging assembly 112 is rigidly secured relative to the positioning element 118. This rigid relationship ensures that the portion of the sensing device 110 that is visible in the images obtained by the imaging assembly 112 is always static relative to the image. This static nature of the imaged sensing device 110 is beneficial in that a single transformation can be employed to transform spatial features of the sensing device from the real-world space to the image space. In particular, since the anatomical location (e.g. sternal notch) that uniquely mates with the positioning element is static in the images, the location of the anatomical feature on the patient is also static. Accordingly, the location of the anatomical feature is known in the image, based on a pre-computed determination of the location of the positing element within the image. This static nature of the imaged portion of the sensing device 110 is evident in FIG. 3D, which shows an image obtained in the absence of a user, and in FIG. 3F.

As shown in FIGS. 3A-3C, the sensing device 110 is operatively coupled to the base 120. In some embodiments, the base 120 may include a base housing (not shown) that includes grooves, notches, cavities, platforms, etc. or is otherwise, generally shaped to allow a portion of the housing of the sensing device 110 to be disposed thereon in a first configuration (e.g., a storage configuration, or charging configuration, in which the sensing device 110 is not being used). In some embodiments, the base housing of the base 120 may include a platform projecting, protruding, coupled to, or otherwise, formed in the base housing on which the sensing device 110 may be disposed in the first configuration.

In some embodiments, the base housing may include or define one or more grooves or cavities within which the positioning element(s) 118 may be disposed in the first configuration. For example, the base housing may define a first groove or cavity configured to receive a first position element 118 of the sensing device (e.g., the spherical portion included in a sternum alignment tool), and a second groove or cavity configured to receive a second positioning element (e.g., a second protrusion or arm coupled to or monolithically formed in the sensing device housing) in the first configuration. In some embodiments, the base housing may also include an arm that may define one or more grooves, and that may be configured to receive and/or support a portion of the sensing device housing in the first configuration. In use, the sensing device 110 can be removed from the base 120 and be placed on a patient, for example, in a second configuration. In the second configuration, the sensing device 110 can be configured to make the desired measurements of the one or more physiological parameters of the patient.

In some embodiments, the sensing device 110 may also be physically coupled to the base 120 via a linkage assembly (not shown). For example, the linkage assembly may include one or more arms (e.g., an articulating arm) physically coupling the sensing device 110 to the base 120. In some embodiments, the linkage assembly may include a first arm hingedly, pivotally, or otherwise, rotatably coupled to the base housing, and a second arm hingedly, pivotally, or otherwise, rotatably coupled to the first arm at a distal end thereof, and also hingedly, pivotally, or otherwise, rotatably coupled to the sensing device body of the sensing device 110 at proximate end thereof. Such a linkage assembly may provide a wide range of motion (e.g., 360 degrees freedom of motion) to the sensing device 110 to facilitate correct or desired positioning of the sensing device 110 on the body of the patient, while assisting the patient in maintaining the sensing device 110 in a stable position on the torso of the patient. Moreover, communication leads (e.g., electrical lead(s)) may be routed through the one or more arms to communicatively couple the sensing device 110 to the base 120 (e.g., the processor 124 via the communication interface 126), and/or to allow the sensing device 110 to receive electrical power from the base 120.

In some embodiments, the linkage assembly may include one or more conductors, such as, an electrical lead, an electrical wire, a flat cord, a coiled cord, or any other suitable electrical lead physically as well as communicatively coupling the sensing device 110 to the base 120 (e.g., to the processor 124 of the base 120 via the communication interface 126 of the base 120). The electrical lead may be permanently coupled to the sensing device 110 and/or the base 120 or removably coupled thereto. Such an electrical lead may allow complete freedom of motion of sensing device 120 by the patient, thereby facilitating correct positioning of the sensing device 110 on the body (e.g., torso) of the patient as well as increasing portability by reducing weight and mobility of the system 100.

In some embodiments, the sensing device 110 may only be communicatively coupled to the base 120 but not physically coupled to the base 120. For example, the sensing device 110 may include a wireless transceiver (e.g., a half-transceiver, a full-duplex transceiver, an RF transceiver, an optical transceiver, BLUETOOTH® transceiver, a WI-FI® transceiver, a near field communication (NFC) transceiver, any other suitable wireless transceiver or a combination thereof) to send to and/or receive signals from the base 120 (e.g., activation signals, deactivation signals, image or video data signals, accelerometer 114 data signals, sensor(s) 116 data signals, or any other signals pertaining to the operation of the system 100). In some embodiments, the sensing device 110 may include a power source (not shown) and a wireless charging mechanism (e.g., wireless charging coils) configured to receive an electromagnetic charging signal from corresponding wireless charging mechanism (e.g., corresponding wireless charging coils) that may be included in the communication interface(s) 126 of the base 120.

As previously described, the base 120 includes the memory 122, the processor 124, the communication interface(s) 126, and the I/O device(s) 128, and may also include additional components to facilitate operation of the sensing device 110. The memory 122 can be any suitable memory device(s) configured to store data, information, computer code or instructions (such as those described herein), and/or the like. In some embodiments, the memory 122 can be and/or can include one or more of a random access memory (RAM), static RAM (SRAM), dynamic RAM (DRAM), a memory buffer, an erasable programmable read-only memory (EPROM), an electrically erasable read-only memory (EEPROM), a read-only memory (ROM), flash memory, volatile memory, non-volatile memory, combinations thereof, and the like. In some embodiments, the memory 122 can store instructions to cause the processor 124 to execute modules, processes, and/or functions associated with the system 100, such as models, calculations, or other algorithms to analyze image(s) or video(s) captured by the imaging assembly 112, accelerometer 114 data sensor(s) 116 data, etc. In some embodiments, the memory 122 may also be configured to at least temporarily store image and/or video data, accelerometer 114 data, and/or sensor(s) 116 data, for example, until the data is transmitted to a user device or a remote server.

The processor 124 can be any suitable processing device(s) configured to run and/or execute a set of instructions or code. For example, the processor 124 can be and/or can include one or more data processors, image processors, graphics processing units (GPU), physics processing units, digital signal processors (DSP), analog signal processors, mixed-signal processors, machine learning processors, deep learning processors, finite state machines (FSM), compression processors (e.g., data compression to reduce data rate and/or memory requirements), encryption processors (e.g., for secure wireless data and/or power transfer), and/or the like. The processor 124 can be, for example, a general-purpose processor, central processing unit (CPU), microprocessor, microcontroller, Field Programmable Gate Array (FPGA), an Application Specific Integrated Circuit (ASIC), a processor board, a virtual processor, and/or the like. The processor 124 can be configured to run and/or execute application processes and/or other modules, processes and/or functions associated with the system 100. The underlying device technologies may be provided in a variety of component types, for example, metal-oxide semiconductor field-effect transistor (MOSFET) technologies like complementary metal-oxide semiconductor (CMOS), bipolar technologies like generative adversarial network (GAN), polymer technologies (e.g., silicon-conjugated polymer and metal-conjugated polymer-metal structures), mixed analog and digital, and/or the like. In some embodiments, the processor 124 can be configured to receive data from one or more sensors, the imaging assembly, or other components of the sensing device 110 and to process that data, for example, to determine the JVP height or other physiological parameters of a patient. Alternatively or additionally, the processor 124 can be configured to send the data from one or more sensors, the imaging assembly, or other components of the sensing device 110 to one or more remote devices (e.g., via a network or the cloud) for further processing and/or analysis.

The communication interface(s) 126 can be any suitable device(s) and/or interface(s) that can communicate with the sensing device 110 (e.g., any or the devices, sensors, and/or data sources described above with respect to the sensing device 110, and/or any combination or part thereof), a network (e.g., a local area network (LAN), a wide area network (WAN), or the cloud), or an external device (e.g., a user device such as cell phone, tablet, a laptop, or a desktop computer, etc.). Moreover, the communication interface(s) 126 can include one or more wired and/or wireless interfaces, such as, for example, Ethernet interfaces, optical carrier (OC) interfaces, and/or asynchronous transfer mode (ATM) interfaces. In some embodiments, the communication interface(s) 126 can be, for example, a network interface card and/or the like that can include at least an Ethernet port and/or a wireless radio (e.g., a WI-FI® radio, a BLUETOOTH® radio, cellular such as 3G, 4G, 5G, etc., 802.11X Zigbee, etc.). In some embodiments, the communication interface(s) 126 can include one or more satellite, WI-FI, BLUETOOTH, or cellular antenna. In some embodiments, the communication interface(s) 126 can be communicably coupled to an external device (e.g., an external processor) that includes one or more satellite, WI-FI®, BLUETOOTH®, or cellular antenna, or a power source such as a battery or a solar panel. In some embodiments, the communication interface(s) 126 can be configured to receive imaging or video signals from the imaging assembly 112, the movement or positioning signals from the accelerometer or gyroscope 114, and/or sensor data from the sensor(s) 116. In some embodiments, the communication interface(s) 126 may also be configured to communicate signals to the sensing device 110, for example, an activation signal to activate the imaging assembly 112 (e.g., one or more imagers and/or electromagnetic radiation sources included in the imaging assembly 112), the accelerometer or gyroscope 114, and/or the sensor(s) 116.

The I/O device(s) 128 may include any suitable device to receive input from a user or communicate an output to the patient or user. In some embodiments, the I/O device(s) 128 may include an activation mechanism or otherwise, a user actuated element (e.g., a touch button, a push button, a switch, a touchpad, etc.) to turn on or otherwise, activate the sensing device 110, or to allow the user to enter information, request information, or set various parameters of the sensing device 110 (e.g., image capture rate, video bit rate, light intensity, etc.). In some embodiments, the I/O device(s) 128 may include a visual indicator (e.g., LED lights, a display, etc.) to display information to the patient or the user. Such information may include, but is not limited to the patient's JVP, other patient physiological parameters such as patient blood oxygen, heart rate, blood pressure, temperature, etc., time of day, communication interface(s) status (e.g., WI-FI® connectivity status), or any other suitable information or a combination thereof.

In some embodiments, the I/O device(s) 128 may include a microphone. The microphone may, for example, be used to capture the sound of the patient's voice while the patent is speaking. This may be the case where, for example, the base 120 may include appropriate hardware and software to enable a call with another person (e.g., a clinician) at a distance. In addition, or in the alternative, the base 120 may include appropriate hardware and software to provide computer-controlled voice instructions to the patient and to record or otherwise process information spoken back by the patient. In addition, or in the alternative, the microphone (or one of the microphones if the I/O device(s) 128 has multiple microphones) can be in contact with the skin of the patient and used to capture sounds generated by the patient's body, for example, heart sounds (e.g., from the cardiac valves opening and closing), respiratory sounds (e.g., from air moving into and out of the lungs), and/or blood flow sounds from blood following in the blood vessels and/or through the valves. In such embodiments, the microphone may be provided in the sensing device 110 (e.g., be included in the sensor(s) 116 of the sensing device 110). The microphone may be in electronic communication with the processor 126, for example, in order to store the captured sounds and/or transmit them via a communications link.

In some embodiments, the I/O device(s) 128 may include a speaker. In some embodiments this may be the case where, for example, the base 120 includes appropriate hardware and software to enable a call with another person (e.g., a clinician) at a distance. In addition, or in the alternative, the base 120 or the sensing device 110 may include appropriate hardware and software to provide computer-controlled voice and/or otherwise audible instructions to the patient. In addition, or in the alternative, the speaker (or speakers, if more than one) can be used to allow the patient to hear their bodily sounds being captured by the microphone. In some embodiments, the speakers can be used to provide instructions to a user, for example, for positioning the sensing device 110 on the patient for measuring JVP. In some embodiments, depending on signals received from the accelerometer or gyroscope 114, the sensor(s) 116, and/or the imaging assembly 112, the processor 124 of the base 120 can be configured to generate instructions for the user (e.g., patient) to help the user correct an incorrect positioning or usage of the sensing device 110. For example, if the accelerometer or gyroscope 114 provides data to the processor 124, whereby the processor 124 detects that the patient is not at an appropriate angle for measuring JVP, the processor 124 via the speaker or another I/O device 124 can inform the user of such incorrect positioning (e.g., generate a sound, turn on or flash a light, etc.) and/or instruct the user on how to correct his positioning.

The processor 124 may be configured to perform any suitable operations for measuring one or more physiological parameters of a patient and/or communicating such parameters to other remote devices, as previously described in detail with respect to the sensing device 110. In some embodiments, the processor 124 may be configured to communicate an activation signal to the sensing device 110 to activate the sensing device 110, for example, provide electrical power to the various components included in the sensing device 110 in response to the activation mechanism being engaged by the user. In some embodiments, the processor 124 may be configured to receive signals from the accelerometer to determine an angle of inclination or reclining angle of the patient's torso, the location of the imaging assembly 112 relative to the target portion of the body of the patient (e.g., the patient's neck), and/or a rate of displacement or otherwise velocity of the sensing device 110.

In response to determining that the angle of the inclination of the patient's torso is within a predetermined range (e.g., in the range of about 30 degrees to about 60 degrees, inclusive), the target portion of the patient's body (e.g., the patient's neck) is within the field of vision of the imager of the imaging assembly 112, and/or the sensing device is not moving or being displaced at rate that is less than a threshold rate (e.g., less than 0.5 mm/second), the processor 124 may be configured to instruct the imaging assembly to initiate image or video capture of the target portion of the patient's body. In some embodiments, the processor 124 may also be configured to activate the electromagnetic radiation source that may be included in the imaging assembly 112 as previously described herein, to illuminate the target portion of the patient's body, and/or project the reference element(s) on the target portion, as previously described.

While the embodiment of the sensing system 100 depicted in FIG. 3 includes a sensing device and a separate base, it can be appreciated that a sensing system does not need to include a base. For example, as described with reference to FIG. 2, the system 100 can include a sensing device 110 and an optional base 120. Where the system 100 does not include a base, certain components that are contained in the base 120 (e.g., memory 122, processor 124, communication interface(s) 126, and/or I/O device(s) 128 can be included in the sensing device 110.

Referring again to FIG. 2, the control unit 130 can be communicatively coupled to the sensing system 100, for example, including the sensing device 110 and/or the base 120, and configured to generate one or more visual images or elements that can assist in determining the patient's JVP or other cardiorespiratory parameter. The visual images or elements can be generated based on a series of images or video of the neck of the patient received from the sensing system 100. As shown in FIG. 2, the control unit 130 may be separate from the sensing system 100 and may include, for example, a local computer, a user device (e.g., a cell phone, a tablet, a laptop, etc.), a remote server, or a cloud server. In some embodiments, the control unit 130 may be included in the sensing system 100. For example, the components of the control unit 130 may be included or integrated in the base 120 of the sensing system, or instructions corresponding to operations performed by the control unit 130 may be stored on the memory 122 of the base 120, and the processor 124 of the base 120 configured to execute the instructions stored on the memory 124.

Expanding further, the control unit 130 is configured to receive the series of images (or video) of the neck of the patient captured or obtained by the sensing device 110 over a period of time. In some embodiments, the series of images may include image data corresponding to lighting, contrast, color, intensity, phase, amplitude, frequency, image collection time, data, or collection time period, and any other information corresponding to parameters of electromagnetic radiation or optical signal obtained by the sensing device 110 based on reflection from a neck of the patient. The series of images may be obtained by placing a portion of the positioning element(s) 118 of the sensing device 110 in a sternal notch of the patient, with the patient being reclined at a reclination angle (e.g., in a range of about 30 degrees to about 60 degrees). In some embodiments, the image data including the series of images received by the control unit 130 may also include data corresponding to the inclination angle of the patient and motion data corresponding to the motion of the patient (e.g., based on motion data collected by the accelerometer 114 or another sensor coupled to the sensing system 100 and/or control unit 130). The patient may be expected to remain relatively still during the obtaining of the series of images but minor motions due to respiration, swallowing, or talking may be permitted.

As previously described, motion of in the series of images can be attributed to many different physiological movements of the patient. In determining the JVP, the motion of interest is movement of the IJV (or EJV) of the patient due to pressure wave propagation from cardiac contractility. While various systems and methods described herein are described as being associated with movement of the IJV, it should be appreciated that such systems and methods may be additionally, or alternatively, associated with the movement of the EJV. All such variations are envisioned and should be understood to be within the scope of this disclosure.

The pulsation of the IJV, and corresponding portion of the neck of the patient, can be used to determine the JVP of the patient. The signal strength or intensity of the electromagnetic radiation reflected from the neck of the patient and captured by the sensing system 100 can vary with the strength of pulsation of the IJV due to cardiac contraction pressure waves. Visualizing the highest point of pulsation on the neck and recognizing the timing of this pulsation relative to cardiac contraction enables differentiation from arterial pulsations and used to determine the JVP height of the patient. In particular, the signal strength, waveform shape and/or phase relative to cardiac contraction can be used to determine the JVP by the sensing system 100, and generate a visual display (e.g., a digital display on the display 140 or a printed image) indicative of the JVP of the patient, as described herein.

Because the JVP or other cardiorespiratory detection from the neck is based on vascular pulsatile motion, any other motion captured in the series of images may be undesirable and can contribute to noise. In some instances, at least some images of the series of images may include gross motion, for example, due to inadvertent motion of the head or neck of the patient or gross motion of the patient. In some embodiments, the control unit 130 may be configured to detect a gross motion in each of the series of images, which corresponds to motion of the patient (e.g., gross motion of neck or head of the patient) during collection or capturing of each of the series of images. The control unit 130 may be configured to remove a portion of the series of images that have a gross motion greater than a gross motion threshold, for example, based on an intensity, phase, signal, or other parameter of the electromagnetic radiation. In some embodiments, the control unit 130 may be configured to use contiguous images (e.g., contiguous frames in a video) for analysis when one or images are removed from the series of images (e.g., to ensure that there are no gaps in pulsatile motion being measured). In some embodiments, in addition to, or alternately to removing a series of images, the control unit 130 may be configured to use one or more motion stabilization techniques to correct for motion related noise or instabilities.

As described above, in some embodiments, the sensing system 100 can capture a video of a patient's neck. The control unit 130 may be configured to analyze the video to determine gross motion stability using standard motion detection approaches such as optical flow, feature tracking, template matching, etc. Temporal portions of the video with motion above acceptable gross motion threshold can then be removed by the control unit 130. In some embodiments, the control unit 130 may be configured to select the largest segment of contiguous stable images for analyzing and extracting the JVP information for display. The video segment of the video may be sufficiently long to encompass at least 1 cardiac cycle of the patient. In some embodiments, multiple video segments each being sufficiently long can be selected. In some embodiments, the period of time of the contiguous segments that are selected may be in a range of about 5 seconds to about 60 seconds, inclusive of all sub-ranges and values therebetween (e.g., about 5, 10, 15, 20, 25, 30, 35, 40, 45, 50, 55 or 60 seconds, inclusive). In some embodiments, the control unit 130 and/or the sensing system 100 may be configured to use motion stabilization approaches (e.g., optical or digital image stabilization) to improve gross stability of the series of images or videos captured from the patient to increase the number of the series of images or length of video that can be reliably used to determine JVP or any other physiological parameters and/or conditions.

The control unit 130 may also be configured to select one or more images (or to merge multiple images) from the series of images to use as an image 152 to serve as a reference or background image for overlaying additional visual elements and/or information thereon. For example, the image 152 can be used in a composite image 150 that visually indicates the location of the JVP and/or other pseudo-periodic physiological parameter of the patient, as described in further detail herein. Any suitable image of the series of images or any still capture from the video of the patient can be used as long as the selected image has sufficient clarity and resolution to serve as reference or background image on which visual indicators of the patient's pulsatile motion or other physiological parameters are overlaid or superimposed.

In some embodiments, the control unit 130 may also receive the image 152 from a source other than the sensing system 100, e.g., a database, another sensing system, or as an input from a user. For example, in one example implementation, a patient may hold a positioning element to the sternal notch that collects the ECG and has a shoulder rest. The camera may be mounted on the wall (or otherwise secured within a static reference frame that does not move with the patient) and images of both a fiducial secured to the patient and the positioning element may be collected. The distance from the camera to the patient may be calculated using a reference element having known dimensions (e.g. the fiducial, the positioning element, or a separate reference element) and a line drawn from the shoulder rest to the fiducial maybe be employed to generate the region of interest in the image. An illumination source may also be provided with sufficient power to illuminate the neck for generating clear images with the camera. The illumination source could be an infrared source that avoids patient discomfort.

The control unit 130 can also receive synchronizing signal or data that includes at least one synchronizing cycle. In some embodiments, the synchronizing data can include ECG data or an ECG signal of the patient for the period of time during which the series of images or video was obtained, or for the period of time that corresponds to the series of images or video segment that remains after removing a portion of the images that have gross motion above a gross motion threshold, as described herein. In some embodiments, the ECG data of the patient may be measured by external ECG sensors operatively coupled to the control unit 130, and communicated to the control unit 130 directly from the external ECG sensors. In some embodiments, the sensor(s) 116 included in the sensing device 110 may include ECG sensors (e.g., ECG electrodes), which are configured to detect the patient's ECG simultaneously with the imaging assembly 112 capturing images of the patient's neck. Examples of sensing devices including such ECG sensors are described in detail in the '177 application, incorporated above by reference. In some embodiments, the synchronizing signal may include PPG data, that may include a delay to cause the PPG data to be used as a synchronizing signal. In some embodiments, the synchronizing signal may include a respiration signal and the synchronization cycles may include respiratory cycles or sub-cycles incorporating the inspiratory or expiratory phases of the respiratory cycle. The respiratory cycle can be generated, for example, from the image data from the respiratory region of interest. The respiratory ROI is posterior to the neck-line in the supraclavicular region at the base of the neck. This region is also located generally at a vertical height of 1-4 cm ASA depending on the patient angle of inclination. An example external method for generating a respiratory synchronizing signal would employ an accelerometer, which may be incorporated into the sensing device or provided as an external sensor. The accelerometer can detect the rise and fall of the chest. Other methods include using generating chest straps or belts, thoracic impedance, or potentially ECG and/or PPG.

While hereinafter, the various systems and methods are described with respect to ECG data and cardiac cycles, it should be appreciated that the systems and methods described herein are equally applicable to PPG data (e.g., PPG data including a predetermined delay to account for the time different between ventricular contraction and arterial pulsation generating the PPG signal), or respiration data and associated respiration cycles. All such variations are contemplated and should be considered to be within the scope of this disclosure.

The control unit 130 is configured to overlay visual elements on a region of interest 160 of the selected image 152 of the series of images, which is divided into a plurality of spatial segments, nodes, or regions 162 to form a composite image 150. The control unit 130 can be configured to determine intensity information representative of the electromagnetic signal for each spatial segment or node 162 of the plurality of segments or nodes 162. In some embodiments, the control unit 130 is configured to temporally segment waveforms or signals based on a fixed temporal window and to obtain a representative waveform for each node, for example, by averaging the temporally segmented signals. In some embodiments, the control unit 130 can be configured to temporally segment the signal for each node 162 using the ECG signal or any other synchronizing signal. For example, the control until can be configured to define the time window for segmenting the signal as the R-R intervals tied to the ECG signal or as a fixed window around the R-wave or peak of each cardiac cycle of the ECG signal. After temporally segmenting the signal, the control unit can then determine a representative waveform average signal over a number of cardiac cycles for each segment or node 162. It will be understood that the R-wave is but one example feature that can be employed to segment the signal, and that any other feature of the synchronizing signal may be employed in the alternative. For example, any one or more of the R-wave, p-wave, QRS complex, and T-wave may be employed to facilitate segmentation.

The control unit 130 is configured to display the composite image 150, e.g., on a display 140, such that at least one spatial segment or node (subregion) of the region of interest 160 in the composite image 150 includes, or is spatially associated with, an indication 164 (e.g., visual indication or element) associated with or generated based on the representative (e.g., average) signal waveform. For example, according to various non-limiting example implementations, the indication, generated in spatial association with a given subregion, may be a visual representation of the shape of a representative waveform for the subregion, a visual representation of the set of waveform segments pertaining to the subregion, and/or a visual (e.g. quantitative or qualitative) representation of one or more measures characterizing the representative waveform or set of waveform segments, such as, but not limited to, magnitude, phase, noise, or any other parameter characterizing the representative waveform segment 162 or the waveform segments over at least the portion of one or more physiological cycles (e.g. cardiac cycles or respiratory cycles).

While many of the example implementations described herein involve the generation of a composite waveform, based on the processing of waveform segments, prior to the generation of an indication within a composite image, it will be understood that the generation of a composite waveform need not be performed in some alternative implementations. For example, an indication of waveform shape, or an indication characterizing one or more waveform attributes may be generated by processing the set of waveform segments without having first generated a representative waveform.

It will be understood that an indication characterizing a waveform associated with a given subregion need not be rendered, within the composite image, on or over the subregion, but may instead be rendered at another location in the composite image, provided that the indication is spatially associated with the subregion (e.g. via a legend, arrow, or other suitable indication relating the indication to the subregion).

The display 140 may include any suitable display configured to display the composite image such as, for example, an LED display, an LCD display, any other suitable display, or a combination thereof. In some embodiments, the display 152 may be included or integrated with the control unit 130 (e.g., the display of a user device, tablet, laptop or computer associated with the control unit 130). In some embodiments, the display 140 may be included or integrated in the sensing system 100, for example, the base 120 of the sensing system 100. In some embodiments, the display 152 may be separate from the sensing system 100 and the control unit 130. In some embodiments, additionally, or alternately, the control unit 130 may be configured to generate a printed display of the composite image (e.g., on printed paper).

The visual indication or element 164 in each spatial segment or node 162 may be indicative of a physiological parameter of the patient in a portion of the neck of the patient underlying a respective segment or node (subregion) 162 of the region of interest 160. In some embodiments, the indication 164 of the physiological parameter is indicative of pulsatile motion in the portion of the neck of the patient underlying the respective segment or node 162 of the composite image 150.

In some embodiments, the underlying portion of the neck within the region of interest 160 can include the underlying jugular vein JV of the patient, and electromagnetic signals within the spatial segments or nodes 162 can correspond to the pulsatile motion of the JV and therefore be used to determine the JVP of the patient. In some embodiments, the visual indication or element 164 can be representative of intensity information (or average intensity information), e.g., of one or more pixels of a node, which is obtained from video or images captured an imaging assembly.

As shown in FIG. 2, a portion 160a of the plurality of segments 162 that overlay the jugular vein JV include visual indications or signatures that correspond to the pulsatile motion of the JV of the patient. The control unit 130 may also be configured to determine a vertical height of the neck of the patient, and overlay or superimpose one or more height indications indicative of the vertical height of various points on the neck of the patient, as described further below. For example, a first height indication H1, a second height indication H2, and a third height indication H3 can be overlaid on the region of interest 160 of the composite image 150 in some implementations, which serve as height markers on the composite image 150. Thus, a medical professional may use the height indicators to estimate a height of the JVP from the composite image 150. In other words, by being able to view the pulsatile motion of the JV and in knowing the height of the neck, a medical professional may look at the composite image and readily and rapidly determine the JVP of the patient (i.e., the height of the portion of the neck that corresponds to the top of the column of pressure of the JV).

Expanding further, an imager included in the imaging assembly 112 may capture images that include a plurality of pixels. Thus, each image of the series of images is composed of a plurality of pixels. Each pixel can have an intensity that corresponds to the electromagnetic or optical signal strength that is reflected from the surface of the tissue, which is based on the motion and/or other parameters of the underlying portion of the patient tissue, as described herein. In some embodiments, the image collection rate or sampling rate of the video of the patient's neck may be at least two times the frequency of interest included in the electromagnetic or optical signal captured from the neck of patient. In some embodiments, the sampling rate may be at least 15 frames per second. In some embodiments, a sampling rate of the ECG signal obtained from the patient simultaneously with collection of the series of images or video may be at least 100 samples/second.

Each pixel of each of the series of images include intensity information that corresponds to (e.g., proportional to) electromagnetic radiation incident on the imager of the imaging assembly 112 after reflection from the neck of the patient. Thus, a group of pixels located in the same relative location with respect to the neck of the patient in each of the series of images can be viewed as a spatial segment or node of that location of the neck, and thus changes in electromagnetic signal information as reflected in the intensity of the pixels located at the same relative location can be used to track the motion of the portion of the tissue in the that relative location.

As illustrated in FIGS. 3B and 3C, the positioning element(s) 118 of the sensing device 110 can be disposed on the chest of the patient (e.g., at least a portion of the positioning element(s) 118 disposed in the sternal notch SN of the patient) such that the imaging assembly 112 is disposed at a repeatable and fixed position and distance that has a known location relative to the neck of the patient. Based on this location, the control unit 130 may be configured to determine the region of interest 160. For example, the control unit 130 may be configured to outline the region of interest 160 using, at least in part, the location of the sternal notch SN and the known orientation of sensing device 110 relative to the torso of the patient (when the sensing device 110 is appropriately positioned and oriented relative to the patient) as a reference. As explained above with reference to FIGS. 3B, 3C and 3D, the reference location and sensing device orientation does is the same for all images due to the fixed spatial relationship between the imaging assembly 112 and the positioning element(s) 118. This fixed relationship enables the control unit 130 to determine and generate the region of interest 160 at a suitable location where the neck of the user is expected to reside when the sensing device is properly positioned and oriented relative to the patient. While the region of interest is illustrated as a rectangle, it will be understood that the region of interest may take on any suitable shape.

In some example implementations, one or more anatomical features may be detected in the image and employed to generate the region of interest and optionally to facilitate identification of JV pressure waveforms, as described further below. For example, a region of interest may be generated using the earlobe of the patient (detected via image processing) and the sternal notch SN of the patient (known based on contact of the positioning element with the patient). In some embodiments, the control unit 130 may be configured to use feature detection to detect the features of the patient's face and neck (e.g., tip of ear, corner of mouth, eye, nose, etc.) from the series of images and orient the region of interest 160 in the correct orientation relative to the features of the patient. In some example embodiments, it can be beneficial to establish a reference line that closely follows the path of the underlying internal jugular (IJ) vein. This reference line aids in positioning the region of interest, determining or accommodating/compensating for head rotation and associated impact on vertical height, classification of nodes, calculation of vertical height in relation to an anatomical landmark (e.g., the sternal angle), and JVP height assessment either through visual observation or automated detection methods.

The IJ vein typically courses lateral to the medial head of the clavicle, deep to the sternocleidomastoideole, and extends to the region proximate and inferior to the earlobe. This reference line begins from a fixed point (X2, Y2) within the image and extends to either the visible earlobe or local area. The earlobe location in the image can be determined through image processing (segmentation, etc.), neural network analysis, or artificial intelligence techniques. Alternatively, a fiducial marker with predefined size, shape, and distinctive markings can be used to demarcate the termination point, making it easily detectable through image processing methods as mentioned earlier.

Using a fiducial marker offers the added benefit of calculating the relative distance and orientation between the camera and the neck, thereby enhancing the precision of vertical height measurements.

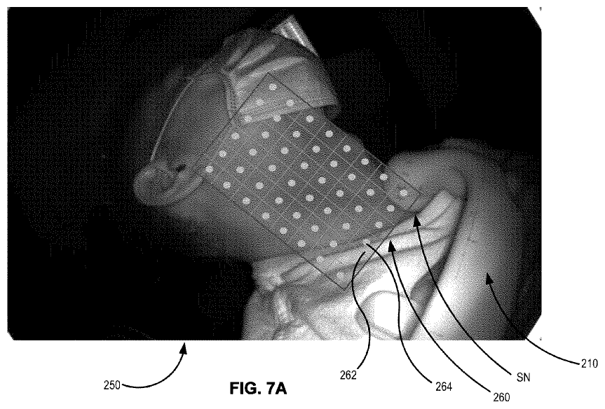

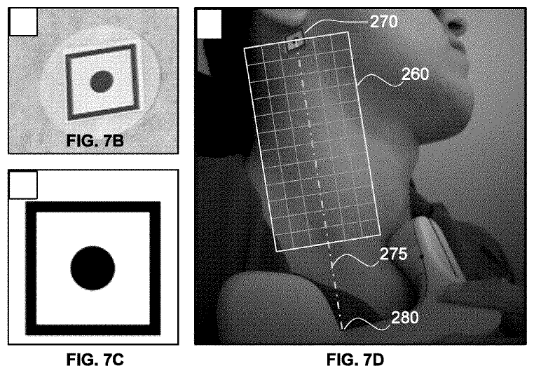

FIGS. 7B and 7C show examples) of fiducial markers that may be employed to identify an earlobe or a local region near an earlobe. FIG. 7D illustrates the determination of the region of interest 260 based on (i) a detected location of a fiducial 270 and (ii) a fixed image location 280. The fixed image location 280 is pre-determined based on a known orientation of the sensing device within the image, such that a reference line 275 extending from the fixed image location 280 to a location of the fiducial 270 represents an expected path of the underlying internal jugular (IJ) vein. In the example implementation shown in the figure, the region of interest 260 is generated such that it is bisected by the reference line. In other example implementations, the region of interest 260 need not be centered on the reference line 275, provided that the reference line 275 passes through the region of interest.

The fixed origin of the line may be located such that the reference line intersects the origin of the sternocleidomastoideond lies along the path of the IJ vein within the image. Alternatively, the fixed location in the image may be determined to lie at or at a prescribed distance relative to a selected location of the sensing device 100 that is captured in the image such that the reference line overlies the origin of the sternocleidomastoideoes along the path of the IJ vein within the image.

The control unit 130 divides the region of interest 160 into the plurality of segments or nodes (subregions) 162, for example, by dividing the rectangle corresponding to the region of interest 160 into a plurality of squares or rectangles having about the same size. The control unit 130 may be configured to use image segmentation to outline exposed skin of the neck, and the nodes 162 may be registered to specific points or subregions on the exposed skin. In some embodiments, the control unit 130 may be configured to use any suitable image segmentation technique such as, for example, region growing segmentation, active contour segmentation, semantic segmentation, instance segmentation, region segmentation, any other suitable segmentation technique, or combination thereof, and may optionally, also use enhanced image segmentation techniques, for example, addition of disparity maps (e.g., stereo vision), depth detection, edge detection, color-based segmentation, etc.

In some embodiments the control unit 130 may be configured to register the nodes 162 (center points within subregions) to the underlying selected image using dots within each region of the neck corresponding to that node 162, with the center of the dot being the registered location (e.g., corresponding to a x1, y1 coordinate; or x2, y2 coordinate). The control unit 130 can be configured to determine the intensity for each node 162 in each contiguous image (e.g., of a video segment or series of images). This results in a two-dimensional (2-D) array of time-varying intensity signals, which each value in the array corresponding to the intensity of the pixel(s) for a node 162.

In some embodiments, for each node, the intensity can be based on the intensity of a single pixel. In some embodiments, for each node, the intensity can be based on a mean intensity of N×M pixels, or a weighted intensity of the N×M pixels. It should be appreciated that spatial averaging over a large area reduces the impact of gross or regional motion, while selecting smaller segmenting regions increases resolution of a specific region but may make the signal more susceptible to gross or regional motion noise. The control unit 130 may be configured to select N×M pixels to comprise a node that provides the best compromise for gross motion noise reduction and resolution. In some embodiments, the calculation of mean intensity can represent the entire width and height of each node 162, or a portion thereof. While the nodes 162 are described herein as being square or rectangular in shape, it can be appreciated that other shapes of nodes (e.g., circular, triangular, etc.) can also be used to generate information for a user. As noted above, the pixel intensity information from the skin is derived from energy reflected from the skin and underlying tissue. The pixel intensity variation relates to tissue motion generated from a variety of sources including, for example, muscle contraction, respiration, underlying vascular pulsation and tissue perfusion.

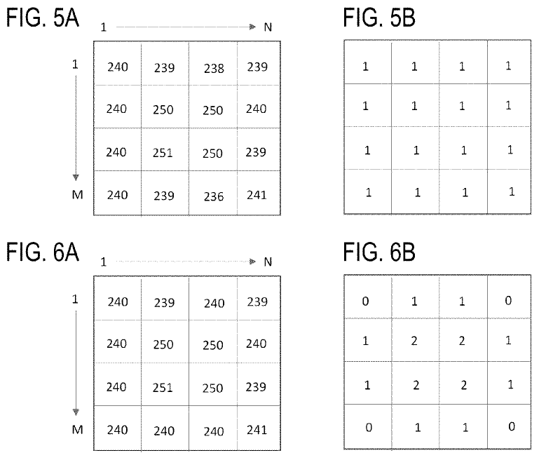

FIG. 5A shows the pixel intensity values of a node of a region of interest, according to embodiments. The mean intensity of the node can be determined by averaging the intensity values of the pixels, with each being assigned the same weight (i.e., of 1 as shown in FIG. 5B). In the embodiment shown, the mean intensity value is 242.FIG. 6A shows the pixel intensity values of a node of a region of interest, according to embodiments. A weighted mean intensity of the node can be determined by assigning weights as shown in FIG. 5B to the pixels and averaging the weighted intensity values of the pixels. In the embodiment shown, the weighted mean intensity value is 245. Other types of weighting or averaging can be used without departing from the scope of the present disclosure.

FIG. 7 schematically depicts the placement of nodes on an image 250 of a neck of a patient, according to embodiments. As described above, a series of images may be obtained by placing a sensing device (e.g., sensing device 110) relative to a sternal notch SN of the patient. In FIG. 7A, a sensing device 210 is depicted. The sensing device 210 may be substantially similar to the sensing device 110 and/or sensing device described in the '117 application. A positioning element (e.g., the positioning element(s) 118) of the sensing device 210 is disposed in a sternal notch SN of the patient. A region of interest 260 can be defined in the image 252. The region of interest 260 is segmented, partitioned, or divided into a plurality of segments or regions 262. Each segment or subregion 262 of the plurality of segments or subregions 262 can be represented as a node. As described above, the node can be registered to the underlying image with its center corresponding to a center 264 of each subregion 262 (e.g., x1,y1; x2,y2; etc.).

Referring back to FIG. 2, in some embodiments, the control unit 130 may be configured to filter the time-varying node signals (e.g., iPPG signals) indicative of the electromagnetic radiation signals or optical signals obtained from the sensing device 110 (e.g., from the series of images or video segments captured by the sensing device 110), to reduce noise. For example, the one or more intensity signals associated with each node may be filtered by the control unit 130 to reduce noise due to gross motion or other artifacts. The intensity signals may be filtered with respect to a frequency range related to the JVP or any cardiorespiratory condition of the patient. Any suitable filtering technique may be used such as, for example, optical filtering or digital filtering, using any suitable filter (e.g., low pass filter, high pass filter, bandpass filter, Fourier transform filter, any other suitable filter or a combination filter). In some embodiments, the intensity signals obtained from the series of images of the patient's neck may include a time-varying signal including a DC (steady state) component and an AC (time-varying) component. The amplitude of the DC component pertains to the steady-state light incident on the imaging assembly 112 of the sensing system 100, while the time varying portion reflects underlying tissue motion (i.e., neck tissue of the patient or other portions of the patient's body captured in the series of images) from a variety of causes as well as underlying vascular pulsations that include jugular vein pulsations that can be used to determine the patient's JVP.

The main categories of pseudo-periodic motion evidence on the neck includes i) respiration ii) venous pulsation and iii) arterial pulsation. Generalized motion associated with speaking, swallowing and head motion is considered non-periodic. The respiratory cycle has an inspiratory and expiratory phase that generates surface motion on the neck in spatially contiguous regions with a general range of 5-40 breaths per minute. Motion from vascular pulsations can be classified as arterial or venous (or a combination of both) and generally ranges between 40-150 beats per minutes. Filtering using well-accepted signal processing techniques (bandpass filter, etc.) can be used to isolate the motion associated with vascular pulsation from that associated with respiration.

In some instances, a large portion of the signal of the non-vascular motion detected by the imaging assembly 112 can have frequency components that are lower than the vascular pulsations. Hence, in some embodiments, bandpass filtering can be employed by the control unit 130 to largely separate motion from underlying muscle and respiratory causes from the higher frequency vascular motions. Bandpass filtering using a passband in a suitable range, for example, between about 0.67 Hz to about 5 Hz, inclusive (corresponding to about 40 beats/minute to about 300 beats/minute heart rate) enables detection of vascular motion, whereas a passband, for example, between about 0.083 Hz to about 0.67 Hz, inclusive (corresponding to about 5 breaths per minute to about 40 breaths per minute) enables detection of respiratory and underlying tissue motion (with the same motion frequency). Thus, the control unit 130 can analyze the image and selectively apply a suitable filter (e.g., a suitable bandpass filter) to determine information corresponding to vascular motion and/or respiratory motion of the patient. It is to be appreciated that some energy from non-vascular motion may have some higher frequency components (harmonics, etc.) that are in the vascular passband and therefore can produce some noise. Such noise can be minimized via further methods described herein (e.g., temporal segmentation and averaging).

In some embodiments, the control unit 130 (or other processing circuitry, including, for example the base 120 of the sensing system 100) is configured to synchronize the time-varying signal for each segment or node measured over the period of time with the ECG signal that was recorded during the same period of time. For example, the control unit 130 may be configured to temporally synchronize the time-varying node signals obtained from the series of images captured by the imaging assembly 112 with an ECG signal captured by an ECG sensor.

In some embodiments in which the sensor(s) 116 of the sensing device 110 includes an ECG sensor, the ECG sensor and the imaging assembly 112 and the ECG sensor may be temporally synchronized by being directed coupled to one another and/or being sampled using the same timing clock or computer. In some embodiments in which the ECG sensor is separate from the sensing device, they can be time synchronized based on time stamps of each of the datasets.

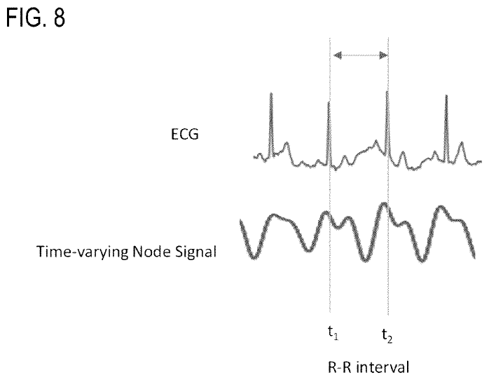

The time-varying electromagnetic signals captured by the imaging assembly (e.g., representative of vascular movements and/or changes) can be periodic, similar to that of the ECG signal. Therefore, by temporally synchronizing the time-varying node signals with the ECG signal, one can use the ECG signal to temporally segment or isolate the cyclical nature of the time-varying node signals. FIG. 8 shows an example of a set of cardiac cycles included in an ECG signal recorded from patient (top plot). R-wave detection techniques can be used to identify the R-waves in each cardiac cycle, and the timing of each cardiac cycle can be captured by the interval between the R-waves of successive cycles (R-R interval). For example, a first R-wave can have a starting time t₁ and a second R-wave can have a starting time t₂, with the difference between t₂ and t₁ representing an R-R interval. The bottom plot shows a time-varying node signal of one node of a region of interest (e.g., a neck), e.g., as obtained from electromagnetic signals captured by the imaging assembly 112. The time-varying node signal, as shown, can be cyclical similar to the ECG signal, as the signal is capturing pulsatile movement of the IJV along with other veins and arteries and therefore is synchronized to the movements of the heart. By time aligning the two signals, one can then temporally segment the time-varying node signal.

As previously described, an external PPG or an internal PPG signal derived from the series of images may be used in addition, or alternatively, to the ECG signals to temporally segment the signals. In some instances, using the ECG signal may be advantageous because the R-wave of the ECG occurs when ventricular contraction occurs. Denoting the timing of lower ventricular (“LV”) contraction relative to the displayed aggregate waveforms has considerable value in estimating the patient's physiological condition. In some embodiments, the timing of LV contraction can also be determined from the PPG with the incorporation of some known delay. For example, PPG signals inherently measure the blood pulsation that takes some amount of time to travel from the heart at LV contraction to the periphery where it is measured (e.g., depending on speed of travel of blood). A predetermined delay time may be subtracted from the PPG signal to account for this delay. While ventricular contraction is typically associated with the QRS complex of the ECG, it may also be determined relative to the p-wave or t-wave.

The control unit 130 may be configured to temporally segment the time-varying signals of each node of a plurality of nodes of a region of interest (e.g., a neck). For example, the control unit 130 may be configured to temporally segment each time-varying node signal into portions or segments using the detected R-waves from the ECG (or a cyclic signal based on PPG such as PPG signal—delay time). In some embodiments, the timing of the ventricular contraction may also be indicated on a composite image as described herein (e.g., associated with each representative waveform or at least a sets of representative waveforms, as described herein). Generally, R-R intervals may vary slightly from one R-R interval to another due to a variety of underlying causes. To account for this, the controller 130 may be configured to map each R-R interval to a fixed temporal window that, in some implementations, may be normalized to facilitate temporal segmentation.

In some embodiments, the control unit 130 may be configured to use data associated with a portion of a cardiac cycle. For example, instead of mapping the R-R interval, the control unit 130 may be configured to use a fixed time duration around the R-wave (e.g., start=t_{R-wave}−0.3 s; duration=1 s). In some embodiments, the control unit 130 can be configured to overlay the temporally segmented node data. In other words, once the time-varying node data has been segmented, each segment of the node data can be overlaid with the other segments. In some embodiments, the control unit 130 may be configured to overlay the temporally segmented node data with the R-wave at the start and end of the cycles, while in other circumstances, the control unit 130 may be configured to overlay the temporally segmented node data with the R-wave in the center or middle. This latter overlaying method may enable better visualization of vascular motion occurring prior to the R-wave (e.g., the a-wave of the JVP that is associated with atrial contraction). The control unit 130 may then be configured to calculate or determine an representative signal from the overlaid temporally segmented node data, as described in further detail in sections below.

The amplitude in the overlaid signals can be absolute or normalized, for example, using maximum amplitude. In some embodiments, the control unit 130 may be configured to perform the normalization across a patient population, across an individual over multiple measurements, spatially across the entire/portion of nodes, or temporally across multiple or continued measurements at a single node/signal. The normalization can be linear or non-linear (e.g., logarithmic or exponential), which can enable accentuation of smaller changes while still capturing the information within large amplitude signals.

In some instances, the time-varying node signal or portions of the time-varying node signal synched with the ECG signal (e.g., using ECG gating) may include one or more unrelated signals (e.g., due to gross motion, swallowing, talking, signal noise, etc.) that are undesirable and may contribute to noise that makes it difficult to determine a reliable mean signal that substantially accurately reflects the underlying physiological parameter of interest (e.g., vascular pulsation and/or respiration) of the patient. In some embodiments, the control unit 130 may be configured to measure the entropy (e.g., level of organization or disorganization) in the temporally segmented node signals for the cardiac cycles within the nodes. The control unit 130 can be configured to perform a single calculation of entropy of the temporally segmented node signals for the cycles within each node, or perform multiple measurements of entropy at different points within the cycles (or at different points within a fixed time window).

It will be understood that entropy can be determined according to several different measurement/processing methods. For example, measuring the variability of the signal at multiple fixed times relative to the R-wave of the signal would enable a measure of entropy. The variability at R-wave+0.50 mS, R-wave+1.0 mS and R-wave+1.5 mS could be calculated for each R-wave and the variability determined.

In some embodiments, a threshold entropy value or range of entropy values can be established (e.g., via user input and/or prior settings), and the control unit 130 may be configured to remove the signals for any node that have entropy that exceeds or is greater than the entropy threshold. In some embodiments, the control unit 130 may display the time-varying node signals (or visual elements corresponding to such signals) that have an entropy less than the entropy threshold or within a range of entropy values and not display such signals (or visual elements corresponding to such signals) that fall outside of the entropy threshold or range. In some embodiments, the control unit 130 may determine the mean time-varying node signal for those nodes that have entropy values that are less than the entropy threshold or within a range of entropy values and not for other nodes.

In some embodiments, the control unit 130 may be configured to display a visual indication or visual element showing the time-varying signal (e.g., overlapping time-varying waveforms) for each (or a subset) of nodes of the plurality of nodes of a region of interest (e.g., a neck), where the time-varying information provides insight into the IVJ pulsation movement and JVP. As described herein, time-varying signals can represent non-temporally segmented signals while waveforms can represent the temporally segment and overlaid waveforms.

For example, as shown in FIG. 2, each physiological parameter indication or visual element 164 for a corresponding spatial segment or node 162 of the region of interest 160 is depicted as a time-varying signal or waveform. Each waveform can be indicative of the motion of the underlying tissue of the corresponding underlying region of the neck of the patient. Each waveform may represent a time-varying intensity signal (or average thereof), as captured from the image or video data of the region of interest 160, displayed in the time domain with the vertical axis representing a value or amplitude of the time-varying intensity signal. The amplitude of the waveform can be relative or absolute. Moreover, as described above, each waveform can be synchronized with a ECG signal of the patient, and the portion that is shown can be for one or more full cardiac cycles or a portion thereof. The start of the horizontal axis can also correspond to the R-wave or another interval of the cardiac cycle (e.g., a fixed internal around the R-wave), for example, to illustrate or indicate the time of ventricular contraction within the waveform corresponding to a cardiac cycle. The latter can enable a medical professional reviewing the composite image 150 to visualize vascular pulsation movements or physiological behavior of the patient prior to ventricular contraction. Displaying the composite image as described herein may provide the benefit of allowing the medical professional or any other observer of the composite image to understand spatial and temporal information of a physiological parameter of interest (e.g., vascular pulsations) of the patient using one or more static images.

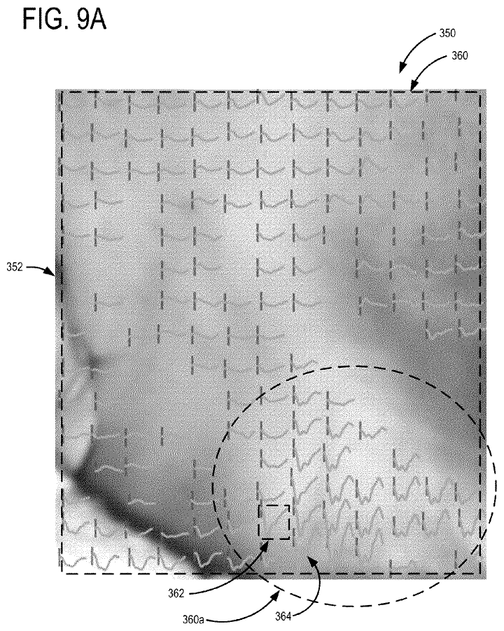

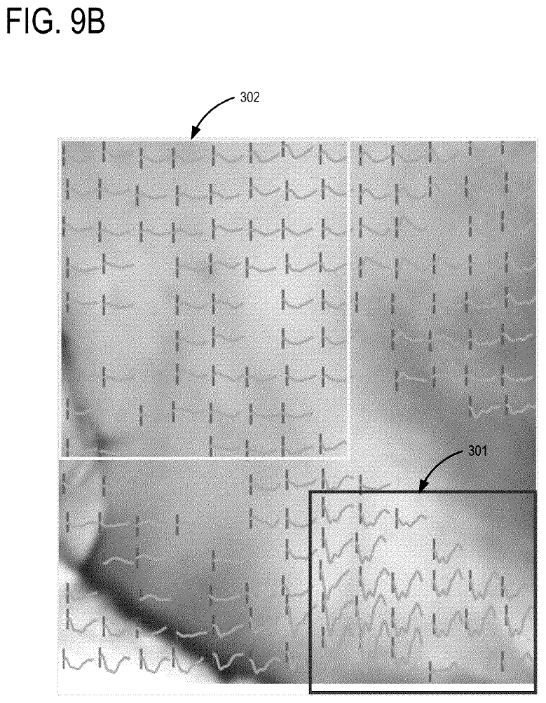

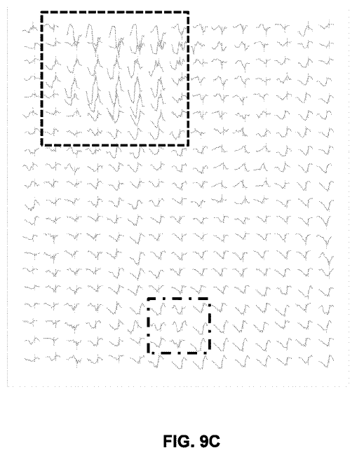

FIG. 9A shows an example static image implemented as a composite image 350 that may be displayed on a display (e.g., display 140). The composite image 350 can be generated by a processor, including, for example, the control unit 130. The composite image includes an image 352 of a portion of a neck of a patient having a region of interest 360. The region of interest 360 is divided into a plurality of segments or nodes 362, each of which is associated with a waveform 364. In some embodiments, the waveforms 364 can be the time-varying signal waveforms for the nodes 362 at each location. As can be seen in FIG. 9A, some spatial segments or nodes 362 of the neck do not include any displayed waveforms. This can be due to the overlapping signal quality (entropy) of the time-varying intensity signals collected for those nodes 362. For example, it can imply that a viable signal was either not received from the corresponding spatial region or area or the signal was too noisy to generate any viable data, and is therefore excluded by the control unit 130 from being displayed in the composite image 350. As previously described, each waveform 364 can represent the time-varying intensity information in one or more images or video that is captured by an imaging device (e.g., imaging device 110). In some embodiments, this time-varying intensity information can be indicative or representative of measurements of tissue motion caused by vascular pressure waves, tissue perfusion or motion. Each waveform 364 can represent a time-varying intensity signal (or average thereof), as captured from the image or video data of the region of interest 160, displayed in the time domain. Where the time-varying signal is indicative of vascular pulsatile motion, the time-varying signal can be synchronized with an ECG signal, and temporally segmented based on cardiac cycle. In some embodiments, a single waveform can be selected from a cluster of waveforms and used as a representative waveform. In some embodiments, the time-varying signal for multiple cardiac cycles can then be averaged, and the waveforms 364 being displayed can represent the average of the temporally segmented signals. In some embodiments, the selection or a representative waveform, or averaging to determine an average waveform to serve as the representative waveform can include performing cluster analysis, as further described below. In FIG. 9A, each waveform 364 can represent the time-varying intensity signal for a particular node that is associated with an entire cardiac cycle, or portion thereof. For example, the waveforms 364 can represent the intensity data within a full cardiac cycle, or they can represent the intensity data during a fixed period within the cardiac cycle (e.g., a fixed period around the R-wave of each cardiac cycle). The vertical line in each waveform segment shown in FIG. 9A can represent the timing of ventricular contraction which can be used as the start of this period, e.g., the R-wave or some fixed point in time prior to the R-wave. In some embodiments, the start of the period can be before, before and after, or just after ventricular contraction. The amplitude of each waveform 364 may be a relative (e.g., normalized) or absolute amplitude. As shown in FIG. 9A, a portion 360a of the region of interest 360 includes a cluster of well-defined waveforms. These well-defined waveforms can have the shape of the JVP waveform and be indicative of the motion of the IJV. Therefore, a physician or other individual can readily observe where these waveforms stop or die down to determine where the top of the column of pressure of the IJV stops and therefore the JVP height. As previously described, synchronization may enable a medical professional to better understand the temporal relationship between waveforms from the neck of the patient and actual cardiac contraction, or respiratory motion.

In some embodiments, the control unit 130 may be configured to reduce noise in the time-varying node signals by implementing waveform averaging, temporal clustering with optional averaging within the temporal clusters of waveforms, and/or spatial clustering of the representative waveforms. Simple averaging can involve detecting the individual cycles in each waveform and normalizing the amplitude and period of all cycles to have the same amplitude and duration. Then, the normalized cycles are averaged to remove the noise. The main drawback of such averaging is that the cycles in each node can have multiple patterns and averaging over multiple patterns may destroy the information content in each pattern. Furthermore, sporadic waveforms may take substantially different shapes due to the presence of ectopic beats such as premature ventricular contractions. As such, in some embodiments, the control unit 130 can implement clustering. For example, the control unit 130 can be configured to reduce noise by clustering the waveforms temporally and/or spatially. Clustering may allow the control unit to select a signal waveform within the clustered waveform to use as the representative waveform, or average at least a portion of the waveforms within the clustered waveforms and use the average waveform as the representative waveform.

Expanding further, the mean signal obtained from the series of images in some instances may misrepresent the underlying physiology of the patient, for example, may include noise from extraneous motion, any other noise signature(s) or a combination thereof. Moreover, averaging the signals over time may not account for phase shifts that can result from extraneous tissue motion despite the actual underlying waveforms or pulsatile motion having similar morphology. In some embodiments, the control unit 130 may be configured to cluster signals based on waveform shape and/or amplitude, and/or may be configured to cluster based on phase shifts. This may provide the advantage of being able to identify morphologically similar waveforms and cluster them into groups depending on their similarity. In some embodiments, the control unit 130 may be configured to use a similarity/distance metric and clustering approach to group similar waveforms.

In some embodiments, the control unit 130 can be configured to perform temporal clustering, or clustering of temporally segmented waveform cycles, and to cluster morphologically similar waveforms. Any suitable clustering approach may be used such as, for example, centroid-based clustering, density-based clustering, distribution-based clustering, fuzzy clustering, partitioning-based clustering, hierarchical algorithmic clustering, any other suitable clustering method or any suitable combination thereof. In some embodiments, the control unit 130 may be configured to use hierarchical agglomerative clustering (HAC) to cluster together similar waveforms. The proportion of similar members that are grouped into a similar cluster can be a clear indicator of repeatability of the underlying signal. Clustered signals that contain less than about 20% of the total cycles are much less likely to be periodic vascular pulsations. Conversely, clustered signals that contain more than about 50% of total cycles are strongly recurring signals. In this manner, the control unit 130 can enable determination of strength of recurring vascular pulsations with outlier removal (e.g., representative of motion artifact).

In some example implementations, clustering can be performed as follows. Each segment can be taken from the time signal and a distance metric can be used (amplitude, morphology and phase) to drive all the segments into clusters that meet a select distance criteria. Subregions (nodes) with multiple clusters (2+) that cannot be grouped because they exceed the distance threshold could be rejected as they have high variability. In other example implementations, AI/machine learning approaches leveraging feature extraction could be employed to derive a set of features that demonstrate low vs high variability of either the waveform segments or the temporally varying signal.

Additionally, or alternatively, the control unit 130 can be configured to perform spatial clustering. In some embodiments, the control unit 130 may be configured to use cluster analysis to group spatially distributed signals with similar shapes. In some embodiments, the control unit 130 may be configured to display or visually identify clustered waveforms having similar shapes, e.g., using different colors (e.g., green, yellow, orange, etc.). FIGS. 9B and 9C depict a visual output based on spatial clustering. With spatial clustering of iPPG signals of the neck, it is likely that the two main groups would represent arterial and venous pulsations, while other nodes may have less similar pulsations and remain unclustered. As depicted in FIG. 9B, the arterial pulsations are marked using a first box 302, and the venous pulsations are marked using a second box 301. In some embodiments, the control unit 130 may be configured to visualize only a single group (e.g., JVP pulsation is not visualized) or multiple groups (e.g., waveform morphology and phase depends on the surface contours of the neck as well as underlying anatomic variations including adipose and muscular tissue).

In some embodiments, the control unit 130 may be configured to apply spatial clustering after extracting the representative signal for each node, as described herein, to signal averaging approaches. Areas without displayed signals on the composite image (e.g., the composite images shown in FIGS. 9A and 9B) can represent clusters (or entropy levels) not meeting the prescribed threshold for similarity or organization. Such an approach to signal clustering has broad-based applications for analysis and display of overlaid physiological signals. In some embodiments, the control unit 130 may be configured to cluster based on features such as waveform upslope, downslope, etc. Data from the cluster analysis with/without timing information regarding ventricular contraction can be fed into the visual display (e.g., for displaying to a user for determining JVP height) or be used to automatically determine the JVP height. The control unit 130 may be configured to generate one or more static images (e.g., composite images) that can display a representation of at least an amplitude (e.g., an average amplitude), waveform, and/or other aspect of the physiological signal for each node of the region of interest over at least a portion of the cardiac cycle. Moreover, data from the clustering analysis along with spatial locations may be stored and used in an artificial intelligence and/or machine learning model to improve determination and display of representative waveforms in a composite image.

In some embodiments, the control unit 130 may also be configured to generate and display heat maps. For example, a static image showing the average waveform for each node or region of a neck of a user may be displayed (e.g., as depicted in FIG. 9A). A user may select one or more nodes or regions in the image of the neck, and the control unit 130 via the display may present heat maps of the underlying waveforms that were averaged to produce the average waveform visible in the static image. In particular, the heat maps can display each of the waveforms corresponding to the cardiac cycles that were used to obtain the representative waveform (e.g., average waveform, a selected waveform from a temporal cluster or an averaged waveform of at least a portion of the temporal waveform cluster) in the static image. The individual waveforms can be overlaid or superimposed on each other for the selected node or nodes, and the heat map can be colored to show the concentration or density of the overlaid waveforms. Such heat maps can preserve individual waveform information (including amplitude, shape, phase, etc.) enabling the observer to easily identify and compare the waveforms and visually determine whether the average waveform is representative of the underlying waveforms. The observer can easily identify signals with low vs high entropy, and can also be configured to include phase and/or amplitude information. Optionally, the heat maps can include one or more vertical bars to show one or more points during the cardiac cycle (e.g., a R-wave's location), e.g., to provide a reference point for assessing the overlaid waveforms. The heatmap or static image 10B can be linked to the overlaid image such that spatial clusters of similar waveforms in the static image have their spatial location highlighted in the overlaid image.

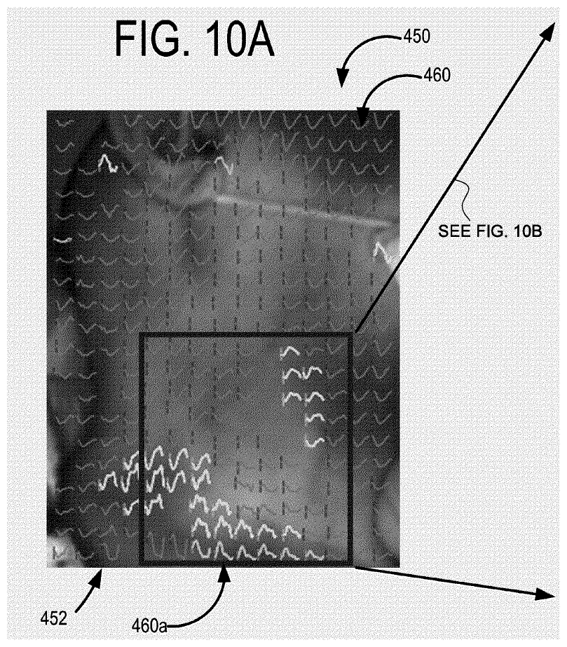

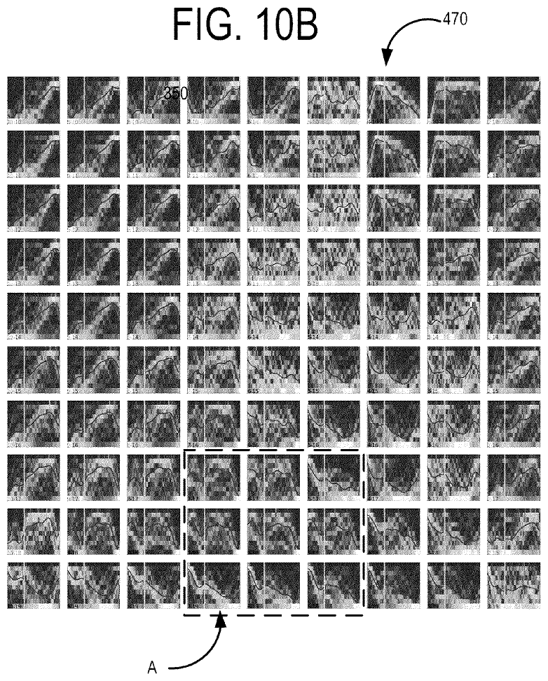

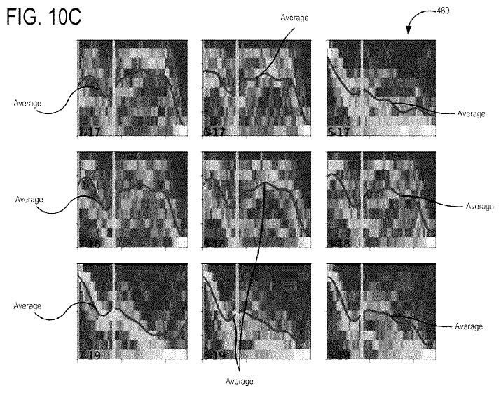

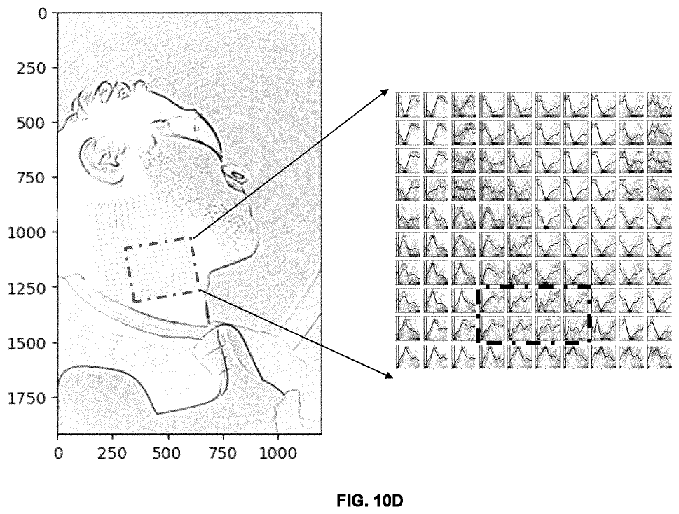

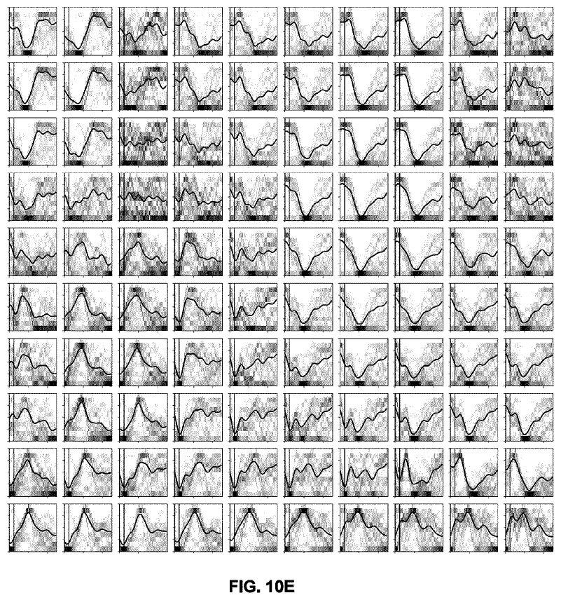

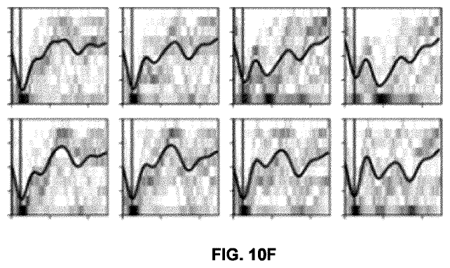

FIG. 10A shows an example composite image 450 of a patient's neck, that includes an image 452 of the patient's neck including a region of interest 460 with visual elements including waveforms being displayed. FIG. 10B shows heat maps 470 corresponding to waveforms included in a portion 460a of the region of interest 460 of the composite image 450. In some embodiments, a user may select the portion 460a to view the heat maps 470. FIG. 10C shows an enlarged view of a portion of the heat maps 470 shown in FIG. 10B, as identified by the arrow A in FIG. 10B. As shown in this enlarged, detailed view, the heat maps 470 display each of the waveforms collected during the cardiac cycles as well as the average waveform. Similar composite images and heat maps are shown in FIGS. 10D-10F.

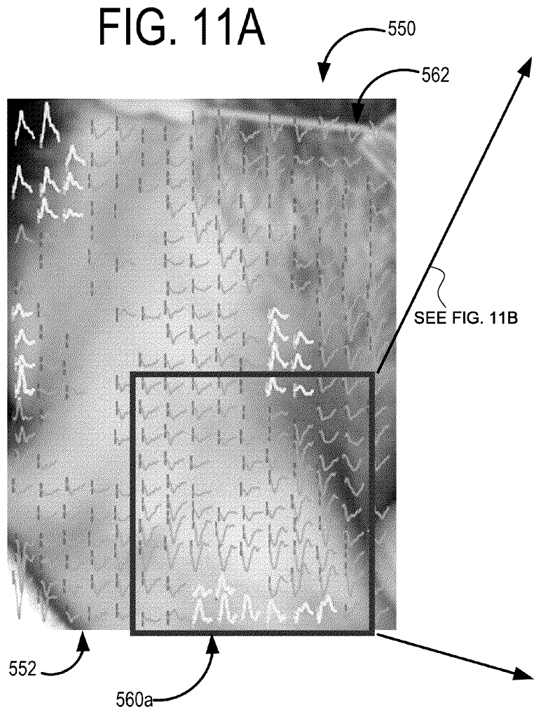

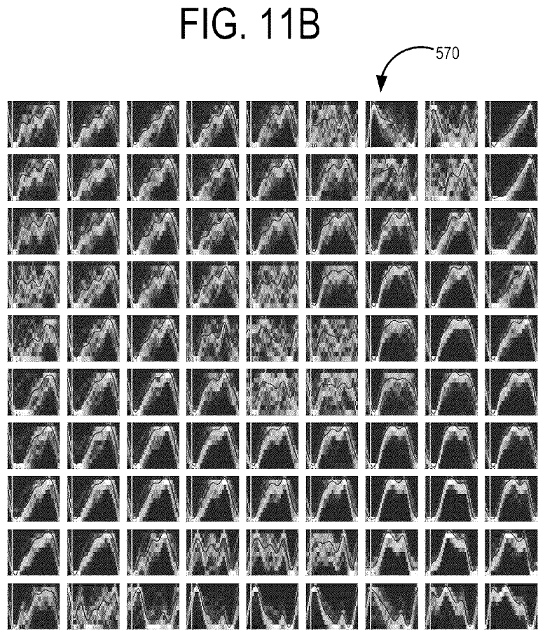

As described previously, the heat maps (e.g., heat maps 470) can allow an observer can easily identify signals with low vs. high entropy so as to determine whether the average signal displayed on the heat map is a reliable signal for determining the JVP or other physiological parameter of interest of the patient. This can allow the observer to assign less weight to or rule out average signals that were calculated based on individual waveforms with too much entropy (e.g., greater than an entropy threshold) or variation. This increases the observer's confidence in using the composite image to estimate the JVP or other physiological parameters of the patient. For example, as seen in FIG. 10A-10B, the individual waveforms displayed in the lower left region of the portion 460a of the region of interest 460 have lower entropy so an observer observing the composite image 450 and the heat maps 470 may have higher confidence that that waveforms in that region of the composite image 450 are representative of the patient's vascular pulsatile motions or other physiological parameter. Similarly, FIG. 11A shows another example composite image 550 of a patient's neck, that includes a selected image 552 of a series of images of the patient's neck having a region of interest 562 with visual elements depicted thereon. FIG. 11B shows heat maps 570 corresponding to waveforms included in a portion 560a of the region of interest 560 of the composite image 550. While not depicted, in some embodiments, representative waveforms depicted in a region of interest can be colored differently based on the level of entropy or variation in their underlying signals. For example, average signals calculated from underlying signals with less entropy (e.g., entropy below a threshold) and greater consistency can be colored a first color (e.g., green), while average signals calculated from underlying signals with greater entropy (e.g., entropy above a threshold) and greater variability can be colored a second color (e.g., red). In some embodiments, a strength of a visual element or indication (e.g., width, thickness) of the representative waveform for each segment or node corresponds to a reliability of the representative waveform that node in representing the individual underlying signals that were used to obtain the representative waveform. For example, lines that are less bold or thick may have individual waveforms with higher variability and therefore less reliability, while lines that are bolder or thicker may have greater reliability.

In some embodiments, the control unit 130 may also be configured to display an indication of vertical height of different locations on the patient's neck on the static or composite image relative to a fixed anatomical reference of the patient (e.g., sternal angle, sternal notch, base of the neck, etc.). For example, as shown in FIG. 2, the composite image 150 may also include the series of height indications H1, H2, and H3, overlaid on the region of interest 160, as previously described herein. This may enable the observer to easily and rapidly estimate the JVP height from the static image. The vertical height lines may be shown at different locations on different composite images and/or be associated with different numbers for different composite images, e.g., depending on the calculation of neck height for each composite image, which can depend on factors including angle of inclination of the patient, pixel scaling of the image, etc.

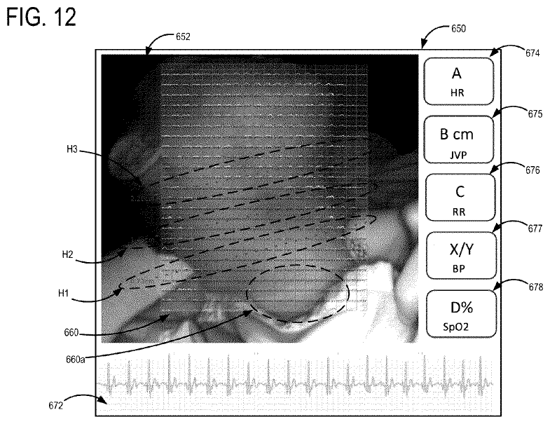

For example, FIG. 12 shows a composite 650 image that may be generated by the control unit 130, according to an embodiment. The composite image 650 includes an image of the neck having a region of interest 660 on which visual elements (e.g., representative waveforms) are overlaid. The region of interest 660 can be divided into a plurality of spatial segments or nodes, each displaying a representative waveform (e.g., selected via clustering and/or time-averaged waveforms) indicative of the vascular pulsatile movement (or cyclical respiratory motion) obtained from optical or electromagnetic signals measured from an underlying tissue portion, as previously described. Moreover, a series of visual height indications or markers H1, H2, and H3 are overlaid on the region of interest that an observer can use to estimate the JVP height. For example, the first visual height indication H1 corresponds to a height of 0.73 cm, the second visual height indication H2 corresponds to a height of 1.46 cm, and the third visual height indication H3 corresponds to a height of 2.20 cm. These are only examples, and different height indicators corresponding to different heights can be displayed on the composite image, for example, based on user input, pre-set settings, etc. Alternatively, or additionally, a larger number of height indication markers (e.g., 4, 5, 6, or even more) or a few numbers of height indicators can be displayed on the composite image.

The primary categories of pseudo-periodic motion observed on the neck include i) venous (JV pulsation) ii) arterial, and iii) respiratory, while general motion associated with activities such as speaking, swallowing, and head movement can be considered non-periodic.

Pseudo-periodic vascular pulsations can be classified as arterial or venous and have a typical range of 40-150 beats per minute. Signal processing techniques enable separation of vascular pulsatile motion from that of respiration and other low frequency motion in the majority of cases. Representative waveforms are generated using synchronized data (eg. R-waves from the ECG) to gate and overlay segments from the time-varying signal. This is followed by a cluster analysis to generate the representative waveforms. Each node can be classified as arterial, venous (JV pulsation) or indeterminant and relies on feature analysis of the representative waveforms and relative timing to the R-wave including an array of spatial and temporal characteristics, including:
i) The JV pulsation aligns closely with a reference line connecting a fixed reference point in the image to a point on the patient, typically the lobe of the visible ear or a fiducial marker with distinct markings near the earlobe, detected through image processing techniques. This reference line approximates the course of the underlying Internal Jugular (IJ) vein, and is described above with reference to FIG. 7D.
ii) JV pulsation typically occurs in close temporal proximity to the R-wave, with slight variations due to underlying conditions such as atrial fibrillation or tricuspid regurgitation. Relative timing is calculated through peak detection applied to the representative waveform at each node.
iii) Arterial pulsations experience temporal delays from ventricular contraction, attributed to the time it takes for arterial blood to travel from the ventricle to the carotid. A method akin to ii) is employed to calculate relative timing.
iv) Timing distribution of pulsations relative to the R-wave is an important feature for classification. Venous pulsations generally occur earlier and are generally out of phase with arterial pulsations with some patient-to-patient variation due to underlying pathology such as severe tricuspid regurgitation and atrial fibrillation. Location of peaks within the timing distribution inform classification of nodes being arterial, venous or indeterminant.
v) Spatial location and proximity of nodes to other nodes with similar waveforms and classification features further supports classification as arterial, venous, or indeterminate nodes. Factors considered for spatial weighting include proximity to similar nodes, proximity to the neckline mentioned in i), and the location of clusters of nodes with higher weighting for groups encompassing the base of the neck.
vi) The JV pulsation waveform exhibits a broader pulsation and larger amplitude compared to the carotid due to the thin-walled, less muscular, and more superficial nature of the IJ vein (and External Jugular, EJ vein). Pulse width, amplitude, and other characteristics are calculated from the representative waveform, encompassing peak height and relative start and end times of the pulsation. The area under the curve provides additional valuable information by combining these features.
vii) The JV pulsation typically displays a slightly more gradual upslope and sharper downslope, while the carotid may have a steeper upslope or a more symmetric pattern. Slopes of the waveforms are computed using peak height, timing, and start-to-end time of the pulsation in the representative waveform.

These features, i)-vi), or a subset thereof, may be weighted and combined to classify nodes as arterial, venous (JV pulsation), or indeterminant. Suitable weights and combinations may be determined experimentally based on waveforms having a known classification (e.g. via clinical adjudication).

In some example implementations, machine learning techniques, such as neural networks or other artificial intelligence methods, can be employed for waveform classification and to determine one or more waveforms that are classified as characterizing JV pulsation. For example, a supervised machine learning approach can be employed, in which a neural network, such as a convolutional neural network or variation thereof, is trained based on waveform shapes being labeled, based on clinical waveform adjudication, as arterial, venous (JV pulsation), or other (e.g. indeterminant). The trained neural network may be subsequently employed for the classification of unknown representative waveforms from the subregions for a given patient. In some example embodiments, one or more of the previously noted features, or other engineered features, may be employed to support machine-leaning-based waveform classification.

Once the classification process is complete, the JVP height is determined as the highest point at which the venous nodes converge along the neck, and this point is compared to a virtual ruler to establish the JVP height above an anatomical landmark like the sternal angle. These principles remain applicable despite heterogeneity across patients and pathology, enabling the classification of jugular venous pulsation and determination of its height.

As shown in FIG. 12, a portion 660a of the visual elements overlays a portion of the neck of the patient where the patient's jugular vein is located. This portion 660a shows representative waveforms that appear to correspond to the shape of a JVP waveform. Based on the location of the top of this portion 660a (or the highest node located in this portion), an observer (e.g., a medical professional) may estimate the JVP height of the patient to be in a range of about 0.4 cm to about 0.5 cm. Thus, the observer may readily and rapidly estimate the patient's JVP based on a single composite image without using complicated diagnostic procedures.

In some embodiments, the control unit 130 may be configured to display other physiological parameters corresponding to the user on the display or as part of the composite image. For example, as shown in FIG. 12, the composite image 650 also displays the ECG recording 672 of the patient obtained during the capture of the series of images or video of the neck. Moreover, the composite image 650 includes a series of panels displaying the patient's average physiological parameters, for example, a first panel 674 displaying the patient's average heart rate (HR), a second panel 675 displaying the patient's estimated JVP height, a third panel 676, displaying the patient's average respiration rate (RR), a fourth panel 677 displaying the patient's average blood pressure (BP), and a fifth panel 678 displaying the patient's average blood oxygen level (SpO₂). These other physiological parameters can be measured during the time period in which the series of images or video of the neck was captured from the patient, or shortly before or after the images or video of the neck were captured. In some embodiments, one or more of the physiological parameters can be captured live and be updated in the display. It should be appreciated that FIG. 12 is only an example composite image, and the control unit 130 may be configured to generate any other suitable composite image including the information as described herein, and optionally, including any suitable combination of other physiological parameters of the patient in any shape or form. For example, rather than having the information of HR, JVP height, RR, BP, etc. all be displayed, only a subset of this information may be displayed. Additionally, this information can be provided in other forms including, for example, visual elements (e.g. text) that are overlaid on the static image, in a table format, etc. All such variations should be considered to be within the scope of the present application.

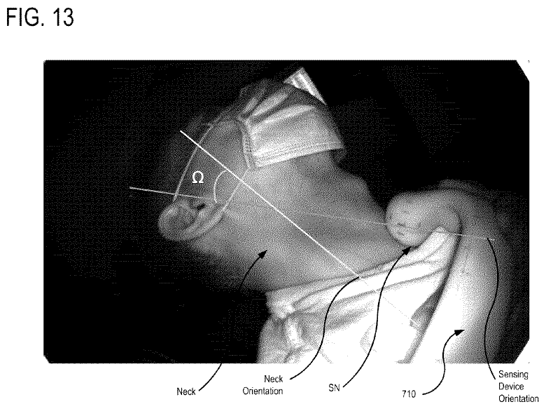

In some embodiments, the control unit 130 may be configured to determine the vertical height of locations on the neck of the patient. For example, the control unit 130 may be configured to determine the vertical height of locations on the neck of the patient and to then generate the vertical height indications in the static images described above based on that determination. In some embodiments, the vertical height determination can be based on an angle of inclination that corresponds to a neck orientation of the patient and/or a sensing device orientation used to capture the series of images of the neck of the patient. For example, FIG. 13 shows an optical image of a neck of a patient having a sensing device 710 of a sensing system disposed on the patient's chest such that at least a portion of the sensing device 710 is disposed in a sternal notch SN of the patient. The sensing device 710 is oriented at an angle Ω relative to a longitudinal axis of the neck of the patient. The angle of the sensing device 710 aligned with the angle of the upper torso may be measured using an accelerometer (e.g., the accelerometer 114 as described with respect to sensing device 110) that can provide an approximate angle of the patient with respect to vertical.

In some embodiments, the inclination angle of the patient may be in a range of about 30 degrees to about 60 degrees, inclusive. However, the image produced by the imaging assembly (e.g., the imaging assembly 112 described with respect to the sensing device 110) can remain largely the same irrespective of angle. Patient anatomy varies considerably from patient to patient and can cause a relative shift between the angle of the patient and the angle of the device. The image on any particular patient remains largely the same with some minor intra-patient variation depending on specific device position (minor), rotation of the neck, etc. As shown in FIG. 13, the line or axis corresponding to the sensing device 710's orientation remains relatively fixed across all angles and the series of images captured, as described above with reference to FIG. 3D, but the line or axis representing the longitudinal axis of the patient's neck may vary across a series of measurements. The sternal notch SN is however, fixed on both the patient, the sensing device 710 (assuming that the patient uses the sensing device 710 in the desired manner with the correct orientation), and location in the image (again as described above). This can enable translation between real world and image coordinate systems—particularly if the circumference(s) of the neck is used in more complex neck models. Hence, patient rotation in the image can be assumed to occur around the sternal notch SN. The control unit 130 may be configured to determine the relative difference between the device angle on the image and longitudinal axis of the neck, i.e., the angle Ω, and use this angle to adjust the angle measured by the accelerometer to improve accuracy

In some embodiments, the control unit 130 may be configured to use segmentation of the skin within the image to determine the longitudinal axis of the neck of the patient within the image (e.g., similar to the segmentation described with respect to node placement). In some embodiments, the control unit 130 may also be configured to incorporate texture mapping, edge detection, any other segmentation method or combination thereof to determine the vertical height.

For example, the control unit 130 may be configured to select a reference point to the left of the sternal notch within the segmented region (e.g., on the neck). Multiple lines intersecting the reference point may be calculated and the minimum distance selected. The longitudinal axis of the neck can be determined by the control unit 130 as being perpendicular to this line. To ensure robustness, multiple reference nodes can be selected. Alternatively, other geometrical shapes can be fit within the segmented region to determine the longitudinal axis of the neck (e.g., square, rectangular, oval, circular, polygonal, etc.)

In some embodiments, the control unit 130 may be configured to determine the vertical height of the neck of the patient by generating a model of the neck of the patient. In some embodiments, the control unit 130 may be configured to model the neck as a 2-dimensional (2D) object, for example, consider the neck to be a 2D object in the image at the depth of the sternal notch. Lines perpendicular to the longitudinal axis of the neck can then be used to identify different vertical heights of the neck.

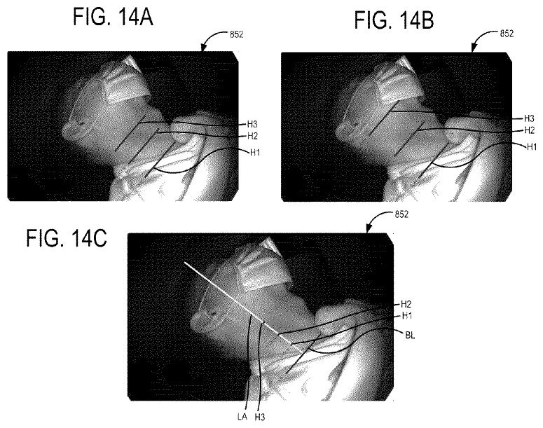

For example, FIG. 14A shows vertical height indicators H1, H2, and H3 superimposed on a selected image 852 of the neck of a patient. The vertical height indications are indicated as lines that are drawn perpendicular to the longitudinal axis of the neck. The vertical height can be estimated by converting between pixels and actual measurement (e.g., centimeters) and the location z_{rw}=0 corresponding to the location of the sternal notch can be known and used to calculate vertical measurement locations assuming that the sensing device is placed with the sternal notch throughout capturing of the series of images. Provided that the imaging assembly of the sensing device is fixed relative to the positioning element, and that the sensing device is configured to facilitate orientational alignment with the patient (e.g. the alignment element has a longitudinal axis and/or multiple anatomical alignment features that facilitate orientational alignment), and also provided that the inclination angle of the patient is known, a calibration can be determined between image pixels and actual physical height. The control unit 130 may be configured to use standard trigonometry calculations to translate virtual height on the image 852. The values of the vertical height indicators may change based on the inclination or reclination angle of the patient. While FIGS. 14A-14C illustrate the generation of height markers perpendicular to a computed axis of the neck, the height markers may alternatively be rendered relative to (e.g. perpendicular to) another alignment axis, such as the reference line shown in FIG. 7D.

While FIG. 14A shows the height measurements as fractional numbers, the vertical height markers H1, H2, H3 may be redrawn or spacing therebetween adjusted to represent the height markers H1, H2, and H3 as whole numbers, as shown in FIG. 14B. FIG. 14C shows another visual representation of the visual indicators H1, H2, and H3. In this implementation, the longitudinal axis LA of the neck is displayed on the image 852, and a baseline BL that is perpendicular to the longitudinal axis LA and crosses the sternal notch of the patient is shown as the zero height marker. Then the height indicators H1, H2, and H are displayed as small bars or dots along the longitudinal axis LA.

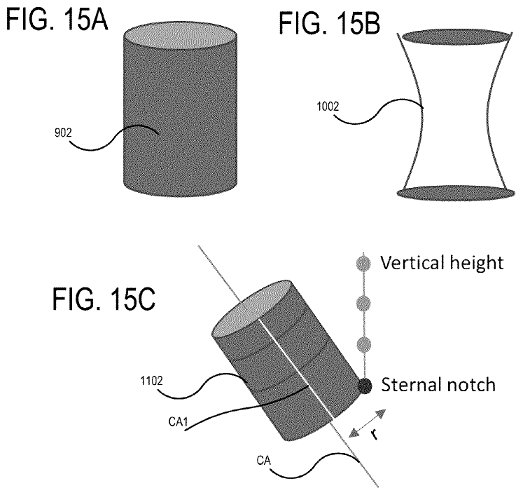

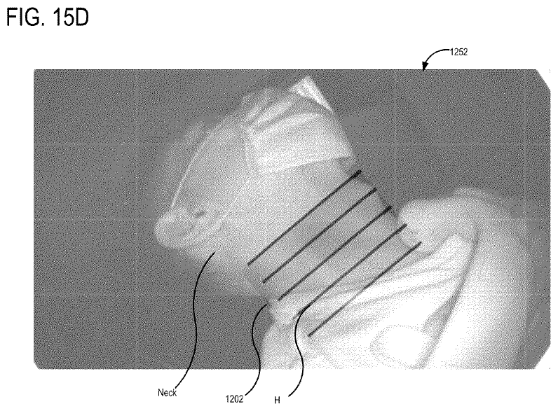

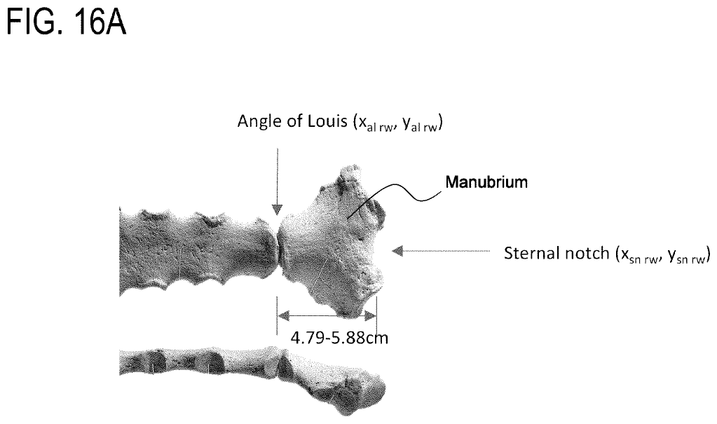

In some embodiments, the control unit 130 may be configured to model as a more complex shape to increase the accuracy of the vertical height measurement. For example, the control unit 130 may be configured to model the neck of the patient as a 3-dimensional (3-D) cylinder by using the known location of the imaging assembly (e.g., the imaging assembly 112) of the sensing device relative to the sternal notch of the patient in 3-D space. FIG. 15A shows a model 902 of the neck of the patient generated by the control unit 130, according to an embodiment. The model 902 models the neck as a 3-D cylinder having a constant uniform cross-sectional width (e.g., diameter) and a length corresponding to a length of the patient's neck. FIG. 15B shows another model 1002 of the neck of the patient generated by the control unit 130, according to another embodiment. The model 1002 models the neck as a 3D cylinder having an hourglass shape. In other embodiments, the control unit 130 may be configured to model the neck of the patient as a conical or trapezoidal cylinder (e.g., tapering inwards from a base of the cylinder to a top surface of the cylinder), or a cylinder having a complex shape that closely mimics the actual shape of the neck of the patient. Assuming the circumference (and hence diameter and radius) of the patient's neck is known (e.g., measured) or estimated from the image (e.g., estimate the transverse distance across the neck, or sternal notch to the ear, then a 3D cylinder or conical with one or multiple diameters can be fit to the long axis into the 2D image of the neck and used for height determination).

The control unit 130 may be configured to determine the location of the one or more height indicators and, optionally, size of the neck using the cylindrical model in any suitable manner. For example, the control unit 130 may be configured to measure the circumference at a midpoint of the cylindrical model when using a simple cylindrical neck model (e.g., the model 902), measuring the circumference at the top, mid-point and bottom of the neck model when the neck is modeled as an hour glass shaped cylinder (e.g., the model 1002) or a conical shape, estimate the neck width and height from the image segmentation, and/or determine width and length of the neck using the series of images captured using an imaging assembly having one or more depth sensing cameras. In some embodiments, the control unit may estimate the size of the neck using manual measurement of the mid-point circumference of the neck or measurement of the circumference of the base, mid-point and top of the neck. Alternatively, the sensing device 110 may include stereo vision or depth sensing cameras, and control unit 130 may be configured to use associated information to determine the size of the neck and fit that to a geometric shape (or use the actual 3-D depth map itself). The control unit 130 may be configured to estimate the circumference/size of the neck by measuring the transverse distance of the neck, the sternal notch to ear, etc. and correlate these measures to different size necks. In some embodiments, the control unit 130 may be configured to mathematically register neck model to the 2D image, as described above.

For example, FIG. 15C shows a model 1102 of the neck that is modeled as simple cylinder by the control unit 130. The radius of the model 1002 is known or calculated from the circumference. The vertical height is projected onto the central axis CA and then projected to the surface of the cylinder (CA1). The approximate coordinate of the pixels at the sternal notch may be known because of the known location of the sensing device at the sternal notch and can also be transformed by the control unit 130 to reflect different depths. This results in surface curves along the neck that vary with the location of the camera in space relative to the sternal notch. FIG. 15D shows a selected image 1252 of a neck of a patient with a cylindrical model 1202 of the patient's neck including multiple vertical height markers H superimposed thereon. While shown as straight lines, the vertical height markets H may be curved lines that conform to a curvature of the neck of the patient, or a curvature of the cylindrical model 1202 of the neck of the patient.

To determine the vertical height indications corresponding to the vertical height of the neck, the control unit 130 may be configured to translate the image coordinate system corresponding to the location of the pixels of the image to real world coordinate system. For example, clinically, the JVP height is typically measured relative to the sternal angle. It can, however, be measured from any static anatomic location on the chest in which the vertical distance to the right atrium is known.

In some embodiments, the control unit 130 may be configured to determine a reclination angle of the patient, and the vertical height determination may be based at least in part on the inclination angle of the patient during collection of the series of images, and an angle of the sensing device relative to a longitudinal axis of the neck of the patient.

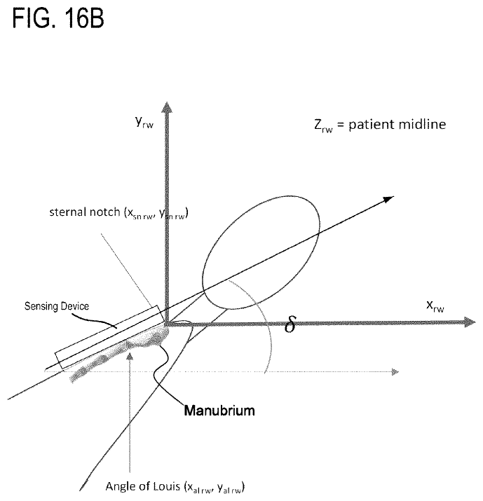

Expanding further, the control unit may be configured to relate the inferior border of the sternal notch to the sternal angle. As described herein, the sternal notch refers to the inferior border of the sternal notch that abuts the manubrium. FIG. 16A shows the spatial relationship between the sternal notch and the sternal angle (angle of Louis), which can play a role in determination of the vertical height indication. The angle of Louis or sternal angle, while often referred to as an angular measure, refers, in the present disclose, to the location of the inferior edge of the manubrium (i.e. the location of the vertex of the angle).

The distance between the sternal angle (angle of Louis) and sternal notch for an adult patient may be in a range of about 4 cm to about 6 cm (e.g., 4.79 cm to 5.88 cm as shown in FIG. 16A). Accordingly, the manubrium length for adult patients can be generally represented as 5.3±0.5 cm. Variance in the size of the manubrium is small and can be ignored for the purposes of this calculation by the control unit 130. The same approach can be used for translation to any other anatomic feature on the chest wall as desired assuming that that the distance to the sternal angle and right atrium are known.

FIG. 16B, shows the sternal notch located in the real world at location (x_{sn rw}, y_{sn rw}, z_{sn rw}) that represent the real world coordinates. The sternal notch is likewise located in the image plane at coordinates (X_{sn image}, Y_{sn image}) that represent the coordinates of the series of images. When the sensing device is disposed in the desired location on the patient's chest, then the real world and image coordinates can be registered, e.g. setting (X_{sn image}, Y_{sn image})=(x_{sn rw}, y_{sn rw}).

Referring again to FIGS. 16B, δ is the angle of sensing device 710 on upper chest of the patient with respect to horizontal, that may be measured by an accelerometer (e.g., the accelerometer 114) included in the sensing device 710, and/or can be based on the inclination/reclination angle of the patient.

In another example implementation, the vertical distance between the right atrium and sternal angle could be measured on CT or MRI images (both are captured with the patient lying flat (horizontal)). This distance can be entered into the system and form a more accurate measure than the addition of the 5 cm vertical height to the H_IJV for all patients. While the 5 cm vertical height is estimated to be typical, this measure however has some variation among patients, and this approach would support a more precise JVP height determination.

In some embodiments, the control unit 130 may be additionally, or alternatively, configured to display a composite image that displays respiratory information of the patient. For example, the control unit 130 may be configured to determine a respiratory motion (e.g., an average respiratory motion, inspiratory phase, expiratory phase, respiratory cycle, each of which are pseudo periodic signals and can be treated very much the same as the pseudo periodic signal from vascular pulsation albeit at a lower frequency) in each segment or node of the region of interest based on the electromagnetic radiation captured in one or more images (e.g., by the imaging device 110), and display in each segment or node of the region of interest, the average respiratory motion for that segment or region. The respiratory motion can be indicative of the patient's respiratory pattern or cycle and respiratory effort.

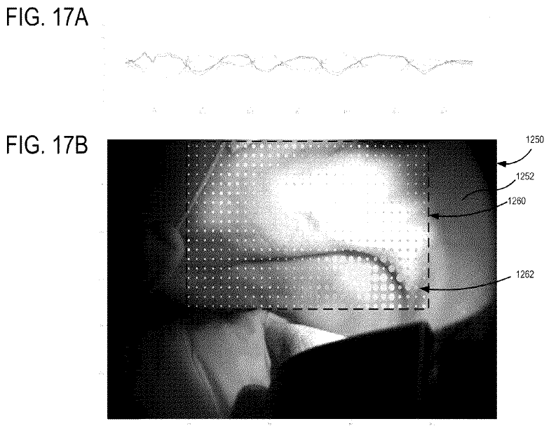

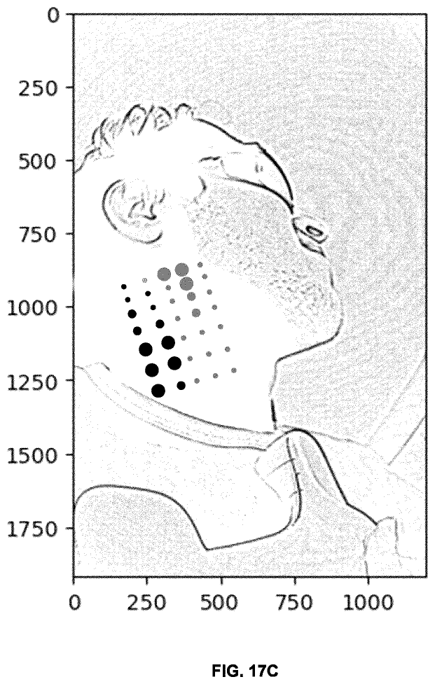

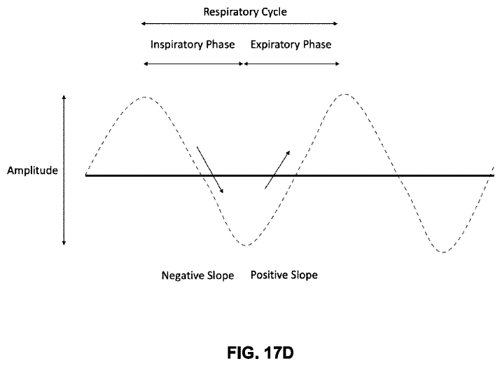

FIG. 17A shows plots of electromagnetic radiation intensity signatures corresponding to respiration movements of the patient obtained from a series of images of a neck of a patient, according to an embodiment. As previously described herein, a passband of the optical signals obtained by band pass filtering, for example, between about 0.083 Hz to about 0.67 Hz, inclusive (corresponding to about 5 breaths per minute to about 40 breaths per minute) can enable detection of respiratory and underlying tissue motion (with the same motion frequency). It will be understood that one could use a range of other passbands as deemed necessary (e.g. 5-60 breaths per minute, or another suitable passband).

In some embodiments, the control unit 130 may use clustering or any other suitable processing method to obtain a representative waveform from the optical signals obtained from the neck of the patient and generate visual elements indicative of respiratory motion that can be overlaid on a selected image of the neck. The image of the neck can be divided into multiple segments, nodes, or areas, with a visual element being generated for each segment or node. The visual element can be generated based on the respiratory waveforms for the spatial segments or nodes of the neck. In particular, the respiratory waveform for each spatial segment or node can be temporally clustered and/or averaged to get mean amplitude and/or phase information corresponding to that spatial segment or node of the neck.

For example, FIGS. 17B and 17C show a composite image 1250 that includes an image 1252 of a neck of a patient and a region of interest 1260 over which one or more visual elements are overlaid, according to an embodiment. The region of interest 1260 can be divided into a plurality of spatially segmented nodes 1262 each including a visual element that depicts an average amplitude and phase of respiration of the patient corresponding to that node of the neck of the patient, according to an embodiment. In the embodiment shown in FIG. 17B, each node 1262 includes a visual element implemented as a circular dot, where the size of each dot represents the average amplitude of the respiration, and a color or shade of each dot (FIG. 17B shows white dots and grey dots) represents an “IN phase” (i.e., inhaling), or an “OUT phase” (i.e., exhaling). However, any other suitable visual indication can be used to display respiration amplitude and/or phase. For example, representative waveforms similar to those described with respect to the JVP signal may be used to indicate the respiratory pattern or effort of the patient. Spatial grouping of the nodes as well as mean amplitude can be used to quantify the degree of indrawing or respiratory distress. This information can be displayed by visually overlaying on the selected image 1252 of the neck of the patient, or used as input in an automatic monitoring system.

Clinically, respiratory effort or work of breathing is assessed by the degree of ‘indrawing’ of the skin of the neck or chest. Because this is a pseudo-periodic signal, overlaying representative waveforms of respiratory effort can enable spatiotemporal representation of this physiological signature, which can be beneficial for remote monitoring but as well as quantifying of the degree of respiratory effort. In some embodiments, the control unit may be configured to automatically generate quantification of respiratory effort using the respiratory data in addition to visual interpretation.

Indrawing is a clinical sign that indicates increased respiratory effort or work of breathing. It typically results from airway obstruction or increased resistance to airflow, necessitating the patient to generate higher inspiratory pressures to maintain adequate ventilation and oxygenation. Indrawing manifests as the visible depression of tissue above the clavicles at the base of the neck, just posterior to the sternocleidomastoideole.

The area and depth of the depression associated with indrawing correspond to the degree of inspiratory pressure required to support respiration. Other signs of indrawing include nasal flaring, suprasternal, intracostal, and subcostal indrawing. Indrawing serves as a marker of disease severity for a range of respiratory conditions, including COPD, asthma, bronchiolitis, croup, and other obstructive or restrictive lung diseases.

The examination of pseudo-periodic respiratory motion enables the determination of various respiratory parameters, including respiratory rate, respiratory phase (inspiration/expiration) and duration, and the severity of indrawing. These parameters may be derived from time-varying respiratory signals collected from nodes located at the base of the neck, left the reference line, which constitutes the region of interest for respiratory analysis. The neckline being determined from the fiducial and fixed reference as previously described. The respiratory region is located at a vertical height 1-4 cm ASA (depending on the patient angle) and to the left of the reference line.

Standard signal processing techniques are applied to filter these signals, isolating the pseudo-periodic respiratory patterns, which typically fall within the range of 5-40 breaths per minute. It should be noted that infants and critically ill patients may exceed this range. In this context, a positive slope of the respiratory waveform (shown in FIG. 17D) indicates tissue motion towards the camera, while a negative slope (indrawing) signifies motion away from the camera. Synchronizing data, marking the start of each respiratory cycle, is generated from nodes within the respiratory region of interest. The start of the respiratory cycle is defined as the time of occurrence of the positive peaks (start of inspiratory phase) while the end of the respiratory cycle is the timing of the successive peak (end of expiratory phase), as illustrated in FIG. 17D. Nodes within the respiratory ROI are temporally segmented using the synchronizing data to generate representative waveforms, facilitating the calculation of composite respiratory parameters.

In some example implementations, respiratory parameters may be calculated as follows:
Respiratory rate: This is determined by the duration of the representative waveform, encompassing both inspiratory and expiratory phases.
Respiratory phase and duration: The expiratory phase corresponds to the portion of the representative waveform with a positive slope, and its duration is calculated as the time between the trough and peak. Troughs and peaks, along with their associated times, may be computed using well-established peak detection algorithms. The inspiratory phase corresponds to the portion of the representative waveform with a negative slope, and its duration is calculated similarly to the expiratory phase (see FIG. 17D).
Severity of indrawing: This is quantified by generating a composite score, which accounts for and is based on the area of tissue involved and the depth of tissue depression during inspiration. The amplitude of the representative waveform during the inspiratory phase corresponds to the depth of tissue depression and the degree of indrawing. The area is calculated based on the number of contiguous nodes with representative waveforms in-phase. One example composite score quantifying the severity of indrawing involves summation of all amplitudes across contiguous nodes.

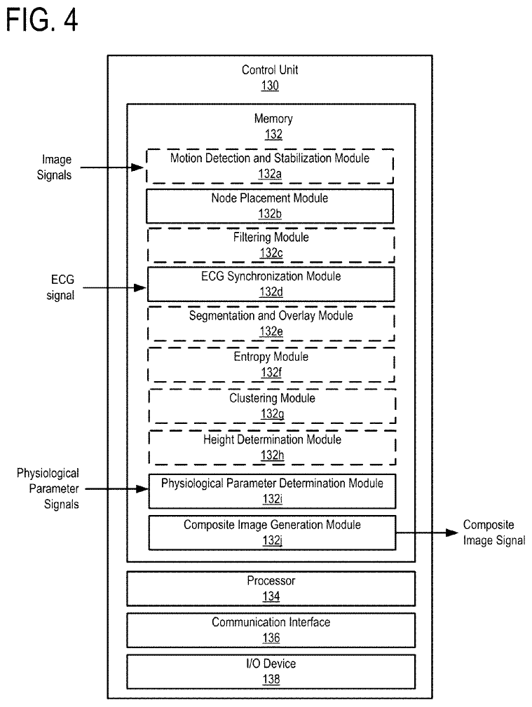

Referring now to FIG. 4, a schematic block diagram of the control unit 130, that is included in the system 10 of FIG. 2, is shown, according to an embodiment. While FIG. 2 illustrates a particular implementation of the control unit 130, any other suitable control unit or controller configured to perform the operations described herein may be used. The control unit 130 can include a memory 132, a processor 134, a communication interface 136, and an input/output (I/O) interface 138. The memory 132 (e.g., Random Access Memory (RAM), Read-Only Memory (ROM), Non-volatile RAM (NVRAM), Flash Memory, hard disk storage, etc.) stores data (e.g., operating parameter data) and/or computer code (e.g., operating parameter filtering or processing algorithms, etc.) for facilitating at least some of the various processes described herein. The memory 132 may include tangible, non-transient volatile memory, or non-volatile memory. The memory 132 may include a non-transitory processor 132 readable medium having stores programming logic that, when executed by the processor 134, controls the operations of the control unit 130. The processor 134 may be implemented as a general-purpose processor, an Application Specific Integrated Circuit (ASIC), one or more Field Programmable Gate Arrays (FPGAs), a Digital Signal Processor (DSP), a group of processing components, or other suitable electronic processing components. In some arrangements, the memory 132 and the processor 134 form various processing circuits described with respect to the control unit 130.

The communication interface(s) 136 can include one or more satellite, WI-FI®, BLUETOOTH®, or cellular antenna. In some embodiments, the communication interface(s) 136 can be communicably coupled to an external device (e.g., an external processor) that includes one or more satellite, WI-FI, BLUETOOTH®, or cellular antenna, or a power source such as a battery or a solar panel. In some embodiments, the communication interface(s) 136 can be configured to receive imaging or video signals, movement or position signals, and/or sensor data from the sensing device 110, as previously described. In some embodiments, the communication interface(s) 136 may also be configured to communicate signals to the sensing device 110, for example, an activation signal to activate an imaging assembly and/or other components of the sensing device (e.g., the imaging assembly 112, the accelerometer or gyroscope 114, and/or the sensor(s) 116). The I/O device 138 may be substantially similar to the I/O device 128 described with respect to the base 120. In some embodiments, the I/O device may include a display (e.g., the display 140) configured to display a composite image including a visual indication of the JVP, the respiration rate, another cardiorespiratory parameter, any other physiological parameter of the patient, or any combination thereof, as previously described.

In some embodiments, the control unit 130 may include various modules implemented in hardware or software configured to perform the operations of the control unit 130. For example, as shown in FIG. 4, the control unit 130 includes a node placement module 132b, an ECG synchronization module 132d, a physiological parameter determination module 132i, and a composite image generation module 132j, and may optionally, also include a motion detection and stabilization module 132a, a filtering module 132c, a segmentation and overlay module 132e, an entropy module 132f, a clustering module 132g, and a height determination module 132h. The processes performed by these modules were described above with reference to the control unit 130.

In one configuration, the motion detection and stabilization module 132a, the node placement module 132b, the filtering module 132c, the ECG synchronization module 132d, the segmentation and overlay module 132e, the entropy module 132f, the clustering module 132g, the height determination module 132h, the physiological parameter determination module 132i, and the composite image generation module 132j can be embodied as machine or computer-readable media (e.g., stored in the memory 132) that is executable by a processor, such as the processor 134. As described herein and amongst other uses, the machine-readable media (e.g., the memory 132) facilitates performance of certain operations of the motion detection and stabilization module 132a, the node placement module 132b, the filtering module 132c, the ECG synchronization module 132d, the segmentation and overlay module 132e, the entropy module 132f, the clustering module 132g, the height determination module 132h, the physiological parameter determination module 132i, and the composite image generation module 132j to enable reception and transmission of data. For example, the machine-readable media may provide an instruction (e.g., command, etc.) to, e.g., acquire data. In this regard, the machine-readable media may include programmable logic that defines the frequency of acquisition of the data (or, transmission of the data). Thus, the computer readable media may include code, which may be written in any programming language including, but not limited to, Java or the like and any conventional procedural programming languages, such as the “C” programming language or similar programming languages. The computer readable program code may be executed on one processor or multiple remote processors. In the latter scenario, the remote processors may be connected to each other through any type of network (e.g., CAN bus, wireless network, etc.).

In another configuration, the motion detection and stabilization module 132a, the node placement module 132b, the filtering module 132c, the ECG synchronization module 132d, the segmentation and overlay module 132e, the entropy module 132f, the clustering module 132g, the height determination module 132h, the physiological parameter determination module 132i, and the composite image generation module 132j may include circuitry components including, but not limited to, processing circuitry, network interfaces, peripheral devices, input devices, output devices, sensors, etc.

In some embodiments, the motion detection and stabilization module 132a, the node placement module 132b, the filtering module 132c, the ECG synchronization module 132d, the segmentation and overlay module 132e, the entropy module 132f, the clustering module 132g, the height determination module 132h, the physiological parameter determination module 132i, and the composite image generation module 132j may take the form of one or more analog circuits, electronic circuits (e.g., integrated circuits (IC), discrete circuits, system on a chip (SOCs) circuits, microcontrollers, etc.), telecommunication circuits, hybrid circuits, and any other type of “circuit.” In this regard, the motion detection and stabilization module 132a, the node placement module 132b, the filtering module 132c, the ECG synchronization module 132d, the segmentation and overlay module 132e, the entropy module 132f, the clustering module 132g, the height determination module 132h, the physiological parameter determination module 132i, and the composite image generation module 132j may include any type of component for accomplishing or facilitating achievement of the operations described herein. For example, a circuit as described herein may include one or more transistors, logic gates (e.g., NAND, AND, NOR, OR, XOR, NOT, XNOR, etc.), resistors, multiplexers, registers, capacitors, inductors, diodes, wiring, and so on.

Thus, the motion detection and stabilization module 132a, the node placement module 132b, the filtering module 132c, the ECG synchronization module 132d, the segmentation and overlay module 132e, the entropy module 132f, the clustering module 132g, the height determination module 132h, the physiological parameter determination module 132i, and the composite image generation module 132j may also include programmable hardware devices such as field programmable gate arrays, programmable array logic, programmable logic devices or the like. In this regard, the motion detection and stabilization module 132a, the node placement module 132b, the filtering module 132c, the ECG synchronization module 132d, the segmentation and overlay module 132e, the entropy module 132f, the clustering module 132g, the height determination module 132h, the physiological parameter determination module 132i, and the composite image generation module 132j may include one or more memory devices for storing instructions that are executable by the processor(s) of the motion detection and stabilization module 132a, the node placement module 132b, the filtering module 132c, the ECG synchronization module 132d, the segmentation and overlay module 132e, the entropy module 132f, the clustering module 132g, the height determination module 132h, the physiological parameter determination module 132i, and the composite image generation module 132j. The one or more memory devices and processor(s) may have the same definition as provided below with respect to the memory 132 and the processor 134.

In the example shown, the control unit 130 includes the processor 134 and the memory 132. The processor 134 and the memory 132 may be structured or configured to execute or implement the instructions, commands, and/or control processes described herein with respect to the motion detection and stabilization module 132a, the node placement module 132b, the filtering module 132c, the ECG synchronization module 132d, the segmentation and overlay module 132e, the entropy module 132f, the clustering module 132g, the height determination module 132h, the physiological parameter determination module 132i, and the composite image generation module 132j. Thus, the depicted configuration represents the aforementioned arrangement in which the motion detection and stabilization module 132a, the node placement module 132b, the filtering module 132c, the ECG synchronization module 132d, the segmentation and overlay module 132e, the entropy module 132f, the clustering module 132g, the height determination module 132h, the physiological parameter determination module 132i, and the composite image generation module 132j are embodied as machine or computer-readable media. However, as mentioned above, this illustration is not meant to be limiting as the present disclosure contemplates other embodiments such as the aforementioned embodiment in which the motion detection and stabilization module 132a, the node placement module 132b, the filtering module 132c, the ECG synchronization module 132d, the segmentation and overlay module 132e, the entropy module 132f, the clustering module 132g, the height determination module 132h, the physiological parameter determination module 132i, and the composite image generation module 132j, or at least one circuit thereof, are configured as a hardware unit. All such combinations and variations are intended to fall within the scope of the present disclosure. In some embodiments, the one or more processors may be shared by multiple circuits (e.g., the motion detection and stabilization module 132a, the node placement module 132b, the filtering module 132c, the ECG synchronization module 132d, the segmentation and overlay module 132e, the entropy module 132f, the clustering module 132g, the height determination module 132h, the physiological parameter determination module 132i, and the composite image generation module 132j) may comprise or otherwise share the same processor which, in some example embodiments, may execute instructions stored, or otherwise accessed, via different areas of memory 132.

The motion detection and stabilization module 132a can be configured to receive image signals from the sensing device 110 and remove or more images of the series of images having a motion above a motion threshold, as previously described herein. The motion detection and stabilization module 132a may also be configured to reduce motion and/or noise associated with motion artifacts in a video of the portion of the body of the patient captured by the sensing device 110, as previously described. For example, once the one or more images, or one or more video segments of a video of the portion of the patient's body have been removed, the motion detection and stabilization module 132a may be configured to perform motion stabilization on the remaining images or the remaining video segment(s).

The node placement module 132b can be configured to outline a region of interest and to divide that region of interest into spatially segmented nodes corresponding to pixels or a set of pixels, as previously described. The filtering module 132c can be configured to filter the signal using one or optical and/or digital filters (e.g., a bandpass filter) to reduce noise, as previously described.

The ECG synchronization module 132d can be configured to receive an ECG signal that is obtained from the patient simultaneously with the capturing of the series of images from the patient, and at least temporarily synchronize the ECG signal with electromagnetic radiation intensity signals from the series of images, as previously described. The segmentation and overlay module 132e may be configured to segment the time-varying node signals obtained from the series of images using the synchronized ECG signals, and overlay the signals obtained from the various cycles over each other, as previously described. The entropy module 132f can be configured to detect signals having an entropy greater than an entropy threshold, and remove such signals from being used in determining an average signal, as previously described.

The clustering module 132g can be configured to use cluster analysis to group signals in the series of images, for example, to further reduce noise and increase signal fidelity, as previously described. The height determination module 132h may be configured to determine the vertical height of the neck of the patient from the series of images and generate visual height indications for overlaying on a composite image, as previously described.

The physiological parameter determination module 132i can be configured to determine spatially segmented and temporally averaged visual indications (e.g., waveforms, dots, etc.) that correspond to vascular pulsatile motion, a JVP height, a respiration rate, or any other physiological parameter of the patient, as previously described, and communicate it to the composite image generation module 132j. In some embodiments, the physiological parameter determination module 132j may also be configured to receive other physiological parameters measured from the patient during, before, or after capturing of the series of images such as, for example, heart rate, respiration rate, blood pressure, blood oxygen level, etc., and also communicate data corresponding to the other physiological parameters to the composite image generation module 132j. The composite image generation module 132j can be configured to generate a composite image signal and to display a composite image on a display (e.g., included in the I/O device 138 or in printed form). The composite image includes an image of a region of interest including one or more of: spatially segmented and temporal indications of the patient's vascular pulsatile motion (e.g., representative waveforms that may be selected from clustered waveforms and/or via averaging as previously described), respiratory motion (e.g., representative waveforms indicative of respiratory motion), mean respiration amplitude and/or phase, height indication superimposed on the selected image, and/or indications of the patient's ECG, PPG data, or other physiological parameters, as previously described.

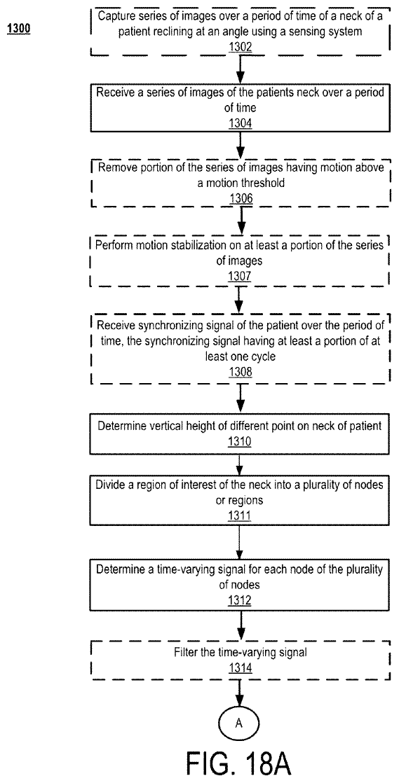

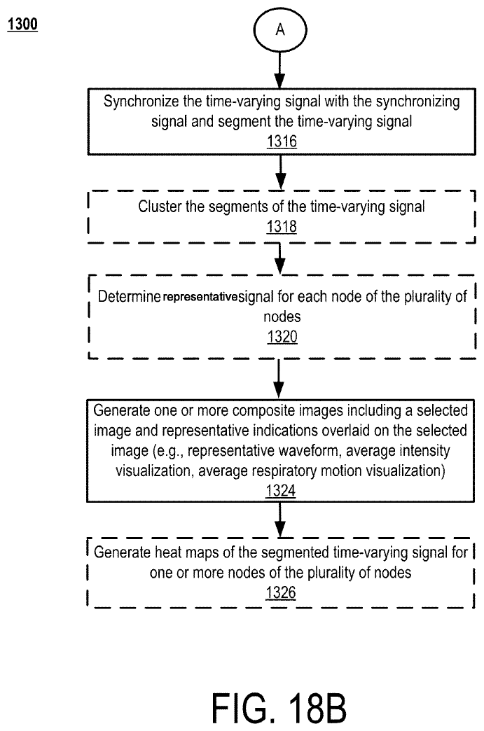

FIGS. 18A-18B show a schematic flow chart of a method 1300 for visually displaying one or more physiological parameters associated with a patient and determined form a series of optical images of a neck of a patient, according to an embodiment. While described with respect to the control unit 130 and the sensing system 100, the operations of the method 130 can be performed with any control unit or controller capable of performing the operations of the method 100 using a series of images collected from any sensing device. All such implementations are envisioned and should be considered to be included within the scope of the present application.

In some embodiments, the method 1300 may include capturing a series of images using the sensing device 110 of the sensing system 100, of a neck of a patient who is lying or sitting in a reclined position at a reclination angle, at 1302, as previously described. At 1304, the control unit 130 receives the series of images of the patient's neck that were captured over the period of time, as previously described. In some embodiments in which the control unit 130 is integrated with the sensing system 100, operations 1302 and 1304 may be performed together in a single operation.

In some embodiments, the method 1300 may also include removing a portion of the series of images, by the control unit 130, which have a motion above a motion threshold, at 1306, as previously described. In some embodiments, the method may additionally or alternately include performing motion stabilization, by the control unit 130, on the remaining portion of the series of images or segment of video, at 1307 as previously described herein.

At 1308, the control unit 130 receives a synchronizing signal of the patient over a period of time, which has at least a portion of at least one cycle. For example, the synchronizing signal may include ECG data of the patient over the period of time that the series of images were captured, or at least the time for which the portion of the series of images were captured by the sensing device 110. The ECG data may include at least a portion of at least one cardiac cycle of the patient, (e.g., a plurality of cardiac cycles) and may be collected using sensors that may be included in the sensing device 110 or using external ECG sensors, as previously described. In some embodiments, the synchronizing signal may include external PPG data, or PPG data extracted from the video or series of images of the portion of the body of the patient captured by the sensing device 110. In some embodiments, the synchronizing signal may include respiration data, as previously described.

The method 1300 may also include determining a vertical height of different points on the neck of the patient by the control unit 130, at 1310, as previously described.

At 1311, the control unit 130 divides a region of interest of the target patient anatomy (e.g., neck) depicted in an image into a plurality of segments or nodes, as previously described. At 1312, the control unit 130 determines a time-varying signal for each segment or node. For example, the control unit 130 may determine the intensity, amplitude and/or phase of the electromagnetic radiation or optical signal within each segment, as previously described.

In some embodiments, the method 1300 may include filtering the time-varying signal, for example, the electromagnetic radiation intensity signal within each segment, at 1314. For example, the control unit 130 may use bandpass filtering or any other optical or digital filtering technique to reduce noise of the signal, and/or to select a particular band of the signal (e.g., corresponding to the JVP signal or the respiration signal of the underlying tissue) for analysis, as previously described. At 1316, the control unit 130 synchronizes the time-varying signal for each segment or node with the synchronizing signal (e.g., the ECG signal) over the period of time and temporally segments the time-varying signal based on the synchronizing signal, as previously described.

In some embodiments, the method 1300 may also include clustering the temporally segmented signal waveforms for each segment or node, for example, to cluster signals having morphologically similar waveforms and remove other signals, at 1318, as previously described. In some embodiments, the method 1300 may include determining an averaging the signals for each segment or node to obtain an average node signal (e.g., a representative waveform), at 1320 as previously described.

At 1324, the control unit 130 generates a composite image that includes an image of the target anatomy having a region of interest with visual elements overlaid thereon. The region of interest may include spatially segmented representative indications (e.g., a selected waveform from clustered waveforms, and temporally averaged waveforms, clustered and temporally averaged waveforms, dots, bars, etc.) of the patient's physiological parameter, for example, the patient's vascular pulsatile motion, respiratory motion, or other physiological parameter, as previously described. In some embodiments, the composite image may also include visual height indications corresponding to the height of different locations on the patient's neck and that can be used by an observer (e.g., a medical professional) to estimate a JVP height of the patient from the composite image, as previously described. In some embodiments, the composite image may also include visual indications of physiological parameters of the patient such as, for example, the patient's ECG data, respiration rate, JVP value, heart rate, blood pressure, blood oxygen level, any other patient physiological parameter, or combination thereof that were collected from the patient during, before, or after capturing of the series of images from the patient. In some embodiments, the method 1300 may also include determining, by the control unit 130 based on the average signal strength in each segment, an average respiratory motion in each segment or node of the region of interest. In such embodiments, the method 1300 may also include generating a respiratory motion composite image, at 1324. The composite image may include an image of a region of interest (e.g., a neck) with visual elements indicative of the respiratory motion overlaid on the selected image, the region of interest being segmented into a plurality of segments or nodes, as previously described.

Optionally, the method 1300 may also include generating heat maps of the underlying waveform signals that were used to determine the average signals displayed within each segment or node of the region of interest, e.g., based on a heat map signal generated by the control unit 130, at 1326, as previously described.

In some embodiments, the patient monitoring described herein can be performed outside of a clinical setting, while in other embodiments, such monitoring can be performed in a hospital or clinical setting, or in other suitable settings.

Indeed, in some example implementations, a clinician need not be present during the monitoring session and/or the monitoring session need not occur in a clinical setting. In such cases, the monitoring process can be initiated and repeated by the patient, e.g., according to a schedule prescribed by the clinician.

As described above, the design of the sensing device enables correct positioning and orientation of the device relative to the patient, such that the sensing device may be employed in a home use setting, remote from a clinician. The image processing steps performed to generate a composite image suitable for inferring JVP height, or performed to autonomously determine the JVP height, need not occur at the location where the monitoring session takes place (e.g., at a patient's residence), or may partially occur at the location where the monitoring takes place. In some example implementations, a patient may be notified that a measurement was correctly performed, but prevented from viewing the composite image and/or JVP result prior to review by a clinician.

The captured video (image series) may be transmitted by any suitable methods and/or devices to a location where it may be processed to generate the composite image and/or autonomously determine the JVP. In view of this, monitoring sessions may occur with a much greater frequency than would be the case if the patient were required to travel to a clinical setting for each monitoring session. For example, the image series and synchronizing data (and/or other partially processed data, such as an extracted time-dependent signal for each subregion) may be transmitted to a remote location (e.g. a server) for further processing. A clinician can then access the remotely processed and/or stored composite image or JVP result by any suitable means; e. g., via a communication interface (e.g., communication interface 126), via the Internet if the device 500 is so configured and is connected to the Internet, via the image series extraction from the device 500, via a smartphone (or other device) and then subsequent transmission to the clinician over the Internet, via the video's extraction from the device 500 on a USB key which is then physically given to or sent to the clinician for analysis, etc. The present technology is not limited in its application to the details of construction and the arrangement of components set forth in the preceding description or illustrated in the drawings. The present technology is capable of other embodiments and of being practiced or of being carried out in various ways. Also, the phraseology and terminology used herein is for the purpose of description and should not be regarded as limiting.

Thus, particular implementations of the invention have been described. Other implementations are within the scope of the following claims. In some cases, the actions recited in the claims can be performed in a different order and still achieve desirable results. In addition, the processes depicted in the accompanying figures do not necessarily require the particular order shown, or sequential order, to achieve desirable results. In certain implementations, multitasking and parallel processing may be advantageous.

It should be noted that the term “example” as used herein to describe various embodiments or arrangements is intended to indicate that such embodiments or arrangements are possible examples, representations, and/or illustrations of possible embodiments or arrangements (and such term is not intended to connote that such embodiments or arrangements are necessarily crucial, extraordinary, or superlative examples).

The use of “including”, “comprising”, or “having”, “containing”, “involving” and variations thereof herein, is meant to encompass the items listed thereafter as well as, optionally, additional items. In the description the same numerical references refer to similar elements.

It must be noted that, as used in this specification and the appended claims, the singular form “a”, “an” and “the” include plural referents unless the context clearly dictates otherwise.

As used herein, the term “about” or “generally” or the like in the context of a given value or range (whether direct or indirect, e.g., “generally in line”, “generally aligned”, “generally parallel”, etc.) refers to a value or range that is within 20%, preferably within 10%, and more preferably within 5% of the given value or range.

As used herein, the term “and/or” is to be taken as specific disclosure of each of the two 10 specified features or components with or without the other. For example, “A and/or B” is to be taken as specific disclosure of each of (i) A, (ii) B and (iii) A and B, just as if each is set out individually herein.

Modifications and improvements to the above-described implementations of the present technology may become apparent to those skilled in the art. The foregoing description is intended to be exemplary rather than limiting. The scope of the present technology is therefore intended to be limited solely by the scope of the appended claims.

In the context of the present specification, the words “first”, “second”, “third”, etc. have been used as adjectives only for the purpose of allowing for distinction between the nouns that they modify from one another, and not for the purpose of describing any particular relationship between those nouns. Thus, for example, it should be understood that the use of the terms “first unit” and “third unit” is not intended to imply any particular type, hierarchy or ranking (for example) of/between the units. Nor is their use (by itself) intended to imply that any “second unit” must necessarily exist in any given situation.

As utilized herein, the terms “substantially’ and similar terms are intended to have a broad meaning in harmony with the common and accepted usage by those of ordinary skill in the art to which the subject matter of this disclosure pertains. For example, the term “substantially flat” would mean that there may be de minimis amount of surface variations or undulations present due to manufacturing variations present on an otherwise flat surface. It should be understood by those of skill in the art who review this disclosure that these terms are intended to allow a description of certain features described and claimed without restricting the scope of these features to the precise arrangements and/or numerical ranges provided. Accordingly, these terms should be interpreted as indicating that insubstantial or inconsequential modifications or alterations of the subject matter described and claimed are considered to be within the scope of the inventions as recited in the appended claims.

The terms “coupled,” and the like as used herein can refer to the joining of two members directly or indirectly to one another. Such joining may be stationary (e.g., permanent) or moveable (e.g., removable, or releasable). Such joining may be achieved with the two members or the two members and any additional intermediate members being integrally formed as a single unitary body with one another or with the two members or the two members and any additional intermediate members being attached to one another. The term “coupling” can also refer to the electrical or electrically communicative coupling of one or more components.

As used herein, the phrase “JVP” refers to the jugular venous pressure. In some example implementations, the JVP may be expressed as a JVP height, relative to an anatomical feature, such as a height measured relative to the sternal angle. In some cases, an additional height offset (e.g. 5 cm) may be added to represent the height offset between the sternal angle and the right atrium. It will be understood that JVP height can be measured from any static anatomic location on the chest in which the vertical distance to the right atrium is known.

The arrangements described herein have been described with reference to drawings. The drawings illustrate certain details of specific arrangements that implement the systems, methods and programs described herein. However, describing the arrangements with drawings should not be construed as imposing on the disclosure any limitations that may be present in the drawings.

It should be understood that no claim element herein is to be construed under the provisions of 35 U.S.C. § 112(f), unless the element is expressly recited using the phrase “means for.”

It should be noted that although the diagrams herein may show a specific order and composition of method steps, it is understood that the order of these steps may differ from what is depicted. For example, two or more steps may be performed concurrently or with partial concurrence. Also, some method steps that are performed as discrete steps may be combined, steps being performed as a combined step may be separated into discrete steps, the sequence of certain processes may be reversed or otherwise varied, and the nature or number of discrete processes may be altered or varied. The order or sequence of any element or apparatus may be varied or substituted according to alternative arrangements. Accordingly, all such modifications are intended to be included within the scope of the present disclosure as defined in the appended claims. Such variations will depend on the machine-readable media and hardware systems chosen and on designer choice. It is understood that all such variations are within the scope of the disclosure. Likewise, software and web implementations of the present disclosure could be accomplished with standard programming techniques with rule based logic and other logic to accomplish the various database searching steps, correlation steps, comparison steps and

It is important to note that the construction and arrangement of the various exemplary embodiments are illustrative only. Although only a few embodiments have been described in detail in this disclosure, those skilled in the art who review this disclosure will readily appreciate that many modifications are possible (e.g., variations in sizes, dimensions, structures, shapes and proportions of the various elements, values of parameters, mounting arrangements, use of materials, colors, orientations, etc.) without materially departing from the novel teachings and advantages of the subject matter described herein. Other substitutions, modifications, changes, and omissions may also be made in the design, operating conditions, and arrangement of the various exemplary embodiments without departing from the scope of the present invention.

While this specification contains many specific implementation details, these should not be construed as limitations on the scope of any inventions or of what may be claimed, but rather as descriptions of features specific to particular implementations of particular inventions. Certain features described in this specification in the context of separate implementations can also be implemented in combination in a single implementation. Conversely, various features described in the context of a single implementation can also be implemented in multiple implementations separately or in any suitable subcombination. Moreover, although features may be described above as acting in certain combinations and even initially claimed as such, one or more features from a claimed combination can in some cases be excised from the combination, and the claimed combination may be directed to a subcombination or variation of a subcombination.

1-28. (canceled)

29. An apparatus, comprising:
a sensing device comprising:
a positioning element configured or structured to facilitate a correct or desired positioning to fit within a suprasternal notch of a patient and facilitate alignment of the sensing device on the body of the patient; and
an imaging assembly rigidly coupled to and spaced from the positioning element, the imaging assembly configured to capture a series of images of at least a portion of a neck of the patient when the positioning element is contacted with the suprasternal notch and aligned with the patient;
processing hardware operatively coupled to the sensing device, the processing hardware comprising a memory and a processor, the memory comprising instructions executable by the processor for performing operations comprising:
receive the series of images of the neck of the patient obtained over a period of time;
receive synchronizing data of the patient over the period of time, the synchronizing data spanning a plurality of cardiac cycles;
define, within the series of images, a region of interest comprising at least a portion of the neck of the patient;
for each subregion of a plurality of subregions defined within the region of interest:
generate a time-dependent signal associated with pixel intensities within the subregion in the series of images;
employ features within the synchronizing data to identify, within the time-dependent signal, a plurality of waveform segments, each waveform segment corresponding to a different cycle; and
process the waveform segments associated with each subregion to identify one or subregions associated with pulsations of an internal jugular vein;
determine, based on user input or sensor data, an inclination angle of the patient;
process at least one of the series of images and the inclination angle to determine a vertical height value corresponding to a convergence of sub-regions associated with pulsations of the internal jugular vein to the highest point along the neck, thereby determining a JVP height.

30. The apparatus according to claim 29 wherein the processing hardware is further configured to:
process at least one of the series of images to identify a fiducial marker residing proximal to an ear lobe of the patient; and
employ the location of the fiducial marker, and a pre-determined reference location, to construct a reference line associated with a location of an internal jugular vein; and
employ the reference line when identifying the one or more subregions associated with pulsations of the internal jugular vein.

31. (canceled)

32. The apparatus according to claim 29 wherein said processing hardware is configured such that processing the waveform segments associated with each subregion to determine one or subregions associated with pulsations of the internal jugular vein comprises:
for each subregion, processing the waveform segments to obtain a representative waveform segment; and
processing the representative waveforms to determine one or more representative waveforms associated with pulsations of the internal jugular vein, thereby identifying one or more associated subregions associated with pulsations of the internal jugular vein.

33-42. (canceled)

43. The apparatus according to claim 29 wherein the processing hardware is configured to generate a composite image comprising:
an image comprising the region of interest;
for at least one subregion:
an indication characterizing at least one attribute of the waveform segments associated with the subregion;
the indication being rendered in spatial association with the subregion; and
the vertical height values, displayed within the region of interest, thereby facilitating a determination of the vertical height corresponding to each indication.

44. The apparatus according to claim 32, wherein the processing hardware is configured such that the representative waveform segment for each subregion is indicative of a physiological parameter of the patient in a portion of the neck of the patient underlying the subregion.

45. The apparatus according to claim 44, wherein the processing hardware is configured such that the physiological parameter is associated with an average vascular pulsatile motion of the patient in the portion of the neck of the patient underlying the respective subregion.

46. The apparatus according to claim 43, wherein the processing hardware is configured such that the indication for each subregion is visually rendered to indicate a degree that the representative waveform segment represents the waveform segments for each subregion.

47. The apparatus according to claim 43, wherein the processing hardware is configured such that for at least one subregion, the indication comprises a visual representation of a phase characterizing the waveform segments associated with the subregion.

48. The apparatus according to claim 43, wherein the processing hardware is configured such that for at least one subregion, the indication comprises visual representation of an amplitude characterizing the waveform segments associated with the subregion.

49. The apparatus according to claim 43, wherein the processing hardware is configured such that for at least one subregion, the indication corresponds to a timing of ventricular contraction relative to the waveform segments associated with the subregion.

50. The apparatus according to claim 43, wherein the processing hardware is configured such that, for at least one subregion, the indication is generated by:
processing the waveform segments associated with the subregion to determine a respective entropy measure associated with each waveform segment; and
excluding a subset of the waveform segments that fail to satisfy entropy criteria when generating the indication corresponding to the subregion.

51. The apparatus according to claim 43, wherein the processing hardware is configured such that, for at least one subregion, the indication is generated by:
processing the waveform segments associated with the subregion to determine a subset of waveform segments satisfying similarity criteria; and
excluding waveform segments that are not members of the subset when generating the indication corresponding to the subregion.

52. The apparatus according to claim 29, wherein the processing hardware is configured such that the vertical height values are based at least on the inclination angle of the patient during collection of the series of images, and an angle of a sensing device determined relative to a longitudinal axis of the neck of the patient.

53. The apparatus according to claim 43, wherein the processing hardware is configured such that, for at least one subregion, the indication comprises a heat map characterizing the waveform segments associated with the subregion.

54. The apparatus according to claim 53, wherein the processor is further configured such that the indication comprising the heat map further comprises a representative waveform characterizing the waveform segments within the subregion.
